(19) United States
(12) Patent Application Publication
(10) Pub. No.: US 2025/0182843 A1
(43) Pub. Date: Jun. 5, 2025

(54) Systems and Methods for Analyzing Genetic Data for Assessment of Gene Regulatory Activity

(71) Applicants: The Trustees of Princeton University, Princeton, NJ (US); The Simons Foundation, Inc., New York, NY (US); The Board of Regents of The University of Texas System, Austin, TX (US)

(72) Inventors: Olga G. Troyanskaya, Princeton, NJ (US); Kathleen Chen, New York, NY (US); Jian Zhou, Dallas, TX (US)

(73) Assignees: The Trustees of Princeton University, Princeton, NJ (US); The Simons Foundation, Inc., New York, NY (US); The Board of Regents of The University of Texas System, Austin, TX (US)

(21) Appl. No.: 18/573,192

(22) PCT Filed: Jun. 21, 2022
(86) PCT No.: PCT/US2022/073065

§ 371 Date: Dec. 21, 2023

Related U.S. Application Data
(60) Provisional application No. 63/362,515, filed on Apr. 5, 2022.
(60) Provisional application No. 63/213,105, filed on Jun. 21, 2021.

(51) Int. Cl. G16B 20/00; G16B 40/20

(52) U.S. Cl. CPC G16B 20/00; G16B 40/20

(57) ABSTRACT

Processes that determine transcriptional regulation from genetic sequence data are described. Generally, computational models are trained to predict transcriptional regulatory effects, which can be used in several downstream applications. Various methods further develop research tools, develop and perform diagnostics, and treat individuals based on identified variants.

### CROSS REFERENCE TO RELATED APPLICATIONS

This application claims priority to U.S. Provisional Application Ser. No. 63/213,105 entitled “Systems and Methods for Analyzing Genetic Data for Assessment of Gene Regulatory Activity,” filed Jun. 21, 2021, and U.S. Provisional Application Ser. No. 63/362,515 entitled “Systems and Methods for Analyzing Genetic Data for Assessment of Gene Regulatory Activity,” filed Apr. 5, 2022, each of which is incorporated herein by reference.

### STATEMENT REGARDING FEDERALLY SPONSORED RESEARCH OR DEVELOPMENT

This invention was made with Government support under Grant No. GM071966 awarded by the National Institutes of Health. The Government has certain rights in the invention.

### FIELD

The disclosure is generally directed to systems and methods for analysis of genetic data evaluation, and more specifically to systems and methods that assess transcription regulatory activity.

### BACKGROUND

Within a typical mammalian genome, the coding DNA (i.e., DNA gene sequences that encode proteins) makes up a very small portion. For example, approximately 2% of the human genome contains sequence that encodes protein. The rest of the genome is noncoding DNA.

Noncoding DNA was long thought to be nonfunctional and often referred to as “junk” DNA. It is now understood, however, that noncoding DNA does in fact have several functions. These functions include regulating gene expression. Noncoding DNA can regulate gene transcription and translation by recruiting various transcriptional and posttranscriptional regulatory factors to a gene via various sequence elements. Various transcriptional sequence elements include promoters, enhancers, CTCF-cohesin binding sites, transcription factor binding sites, polycomb binding sites, heterochromatin sites, and sites of transcription (e.g., transcription start sites, exonic regions).

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1A provides a flowchart of a process to construct and train a computational model to predict a chromatin profile of a genetic sequence in accordance with various embodiments.

FIG. 1B provides a flowchart of a process to utilize a trained computational model to predict the effect of the one or more variants on cis-regulatory activity in accordance with various embodiments.

FIG. 2A provides a flowchart of a process to yield sequence class clusters in accordance with various embodiments.

FIG. 2B provides a flowchart of a process to develop a global, quantitative genetic sequence map of distinct sequence classes of regulation in accordance with various embodiments.

FIG. 3 provides a flowchart of a process to quantitatively assess change in transcription of one or more genes as related to the one or more variants of interest in accordance with various embodiments.

FIG. 4 provides a schematic overview of the Sei model architecture in accordance with various embodiments. 4096 bp sequences, one-hot encoded, are the input to the model (bottom) and the predicted 21,907 cis-regulatory profiles are the output (top).

FIG. 5 provides data graphs depicting Sei model performance on predicting 21907 cis-regulatory profiles on holdout chromosomes in accordance with various embodiments.

FIG. 6 provides heat maps visualizing the rank-transform of pairwise Spearman correlations for the 21,907 cis-regulatory profiles in Sei in accordance with various embodiments. Sei model predictions share a highly similar correlation structure with the experimental observations.

FIG. 7 provides data graphs depicting performance comparison of the Sei model with an earlier model DeepSEA in accordance with various embodiments.

FIGS. 8A to 8C provide a schematic mapping of the global regulatory landscape of genomic sequences, utilized in accordance with various embodiments of the invention. FIG. 8A provides an overview of the sequence classes discovery framework. Sequence classes are extracted from the predicted cis-regulatory profiles of 30 million sequences evenly tiling the genome. The predictions were made by Sei, a new deep convolutional network sequence model trained on 21,907 cis-regulatory profiles from Cistrome database. Specifically, classes are identified by applying Louvain community detection to the nearest-neighbor graph of 180 principal components extracted from the predictions data. FIG. 8B provides a visualization of the global regulatory landscape of human genome sequences discovered by this approach with UMAP. Major sequence classes include cell-type-specific enhancer classes, CTCF-cohesin, promoter, TF-specific, and heterochromatin/centromere classes. FIG. 8C provides a schematic of prediction results. This framework is further applied to predict the sequence class-level genome variant effects, quantified by changes in sequence class scores.

FIGS. 9A to 9C provide heat maps and data graphs showing that sequence classes predict cell type-specific regulatory activities and directional expression-altering variant effects, generated in accordance with various embodiments. FIG. 9A provides a heat map depicting sequence-class-specific enrichment of histone marks, transcription factors and repeat annotations. Log fold change enrichment over genome-average background is shown in the heatmap. FIG. 9B provides Spearman's correlation graphs showing that enhancer sequence classes near transcription start sites are correlated with cell-type-specific gene expression in the applicable tissue or cell types (see Methods). Y-axis shows the Spearman correlation between the proportion of each sequence class annotation within 10 kb to TSS and the tissue-specific differential gene expression (fold over tissue-average). FIG. 9C provides a graph showing that regulatory sequence-class level variant effects are predictive of the directional GTEx variant gene expression effects. The x-axis shows Spearman correlations between the predicted sequence-class-level variant effects and the signed GTEx variant effect sizes (slopes) for variants with strong predicted effects near transcription start sites and the y-axis shows the corresponding log 10 p-values. All colored dots are above Benjamini-Hochberg FDR <0.05 threshold.

FIG. 9D provides a graphical depiction of genome sequence proportion covered by each sequence class, generated in accordance with various embodiments. The proportion of each sequence class is shown in the pie chart. Genome-wide sequence class assignments were based on Louvain clustering of Sei predictions of sequence tiling the genome with 100 bp step size. The clusters unassigned to sequence classes due to the small size (below top 40 clusters) were categorized as “Unassigned.”

FIGS. 10A and 10B provide data graphs showing that variants with strong regulatory sequence class effects show negative selection signatures, generated in accordance with various embodiments. FIG. 10A provides a scatter plot for allele-frequency-based analysis of each sequence class. X-axis shows the average variant allele frequency. Y-axis shows the bidirectional variant effect constraint z-score, which is computed based on logistic regressions predicting common variant (allele frequency >0.01) from sequence-class-level variant effect score for both positive and negative effects. L sequence classes are excluded due to lack of interpretation for their sequence-class-level variant effect scores. FIG. 10B provides comparison of common variant frequencies of 1000 Genomes variants assigned to different sequence classes and variant effect bins. The common variant threshold is >0.01 allele frequency across the 1000 Genomes population. Error bars show+/−1 standard error (SE). The sequence-class-level variant effects are assigned to 6 bins (+3: top 1% positive, +2: top 1%-10% positive, +1, top 10%-100% positive, −3: top 1% negative, −2: top 1%-10% negative, −1, top 10%-100% negative), as illustrated in the bottom right box.

FIG. 10C provides data graphs showing population allele frequency profiles for variants in heterochromatin, low signal, Polycomb, and transcription sequence classes, generated in accordance with various embodiments. Comparison of common variant frequencies of 1000 Genomes variants assigned to different sequence classes and variant effect bins. The common variant threshold is >0.01 allele frequency across the 1000 Genomes population. Error bars show+/−1 standard error (SE). The sequence-class-level variant effects are assigned to 6 bins (+3: top 1% positive, +2: top 1%-10% positive, +1, top 10%-100% positive, −3: top 1% negative, −2: top 1%-10% negative, −1, top 10%-100% negative).

FIG. 11 provides a heat map depicting sequence-class-based partitioning of GWAS heritability, which shows trait associations with tissue-specific regulation, generated in accordance with various embodiments. Partitioned genome-wide heritability in UKBB GWAS with all 40 sequence classes. The size of the dot indicates the proportion of heritability estimated from LDSR, which is conservatively estimated as one standard error below the estimated heritability proportion (bounded by 0). The color of the dot indicates the significance z-score of the fold enrichment of heritability relative to the proportion of all SNPs assigned to the sequence class (bounded by 0). Colored boxes indicate traits associated with blood (red), brain (green), multiple tissues (blue) and promoters (orange).

FIG. 12 provides a polar coordinate graph showing that disease regulatory mutations are predicted to disrupt promoter, CTCF, and tissue-specific regulatory sequence classes, generated in accordance with various embodiments. Sequence-class-level mutation effects of pathogenic noncoding HGMD mutations are plotted. A polar coordinate system is used, where the radial coordinate indicates the sequence-class-level effects. Each dot represents a mutation, and mutations inside the circle with boldest coloring are predicted to have positive effects (increased activity of sequence class), while mutations outside of the circle are predicted to have negative effects (decreased activity of sequence class). Dot size indicates the absolute value of the effect. Mutations are assigned to sequence classes based on their sequences and predicted effects (Methods). Within each sequence class, mutations are ordered by chromosomal coordinates. The associated disease and gene name are annotated for each mutation, and only the strongest mutation is annotated if there are multiple mutations associated with the same disease, gene, and sequence class.

FIG. 13 provides a data graph depicting enrichment Z-score and assessment of heritability in common germline variants that can increase the risk of cancer, generated in accordance with various embodiments.

FIG. 14 provides predicted functional impact of somatic mutations near cancer genes of tumor samples, generated in accordance with various embodiments.

FIG. 15A provides a Kaplan-Meier plot stratifying cancer patients probability of survival based on prediction of impact of somatic variants derived from a cancer sample, generated in accordance with various embodiments.

FIG. 15B provides a Kaplan-Meier plot stratifying cancer patients probability of survival based on prediction of impact of somatic variants derived from a cancer sample in which the variants analyzed are associated with a E5 B-cell-like enhancer class, generated in accordance with various embodiments.

### DETAILED DESCRIPTION

Turning now to the drawings and data, a number of processes for genetic data analysis that can be utilized in various applications, including gene transcription analysis and/or diagnostics in accordance with various embodiments are illustrated and/or described. Numerous embodiments are directed towards frameworks and methods for scoring the impact of genetic noncoding variants on transcriptional regulation. In various embodiments, regulatory elements across a plurality of genomic sites are defined and classified based on their regulatory effect. In some embodiments, the effect of a genetic variant within a regulatory element is determined, which may include determining a first order effect of the variant relative to a reference sequence and/or second order effects on a reference regulatory element. In some embodiments, a genetic variant is analyzed to determine its effect on regulation of one or more transcripts. In some embodiments, a genetic variant of an individual is assessed to determine the impact of the variant on transcriptional regulation, which may be utilized to diagnose the individual. And in some embodiments, an individual can be treated based on diagnostic analysis.

Deciphering how regulatory functions are encoded in genomic sequences is a major challenge in understanding how genome variation links to phenotypic traits. Cell-type-specific regulatory activities encoded in elements such as promoters, enhancers, and chromatin insulators are critical to defining the complex expression programs essential for multicellular organisms, like those affecting cell lineage specificity and development. The majority of disease-associated variants from genome-wide association studies (GWAS) are located in noncoding regions and may perturb regulatory elements, yet without knowing the sequence dependencies of these elements—that is, how changes in sequence affect regulatory activities—one cannot predict the impact of these variants and uncover the regulatory mechanisms contributing to complex diseases and traits. For example, a variant in an embryonic-stem-cell-specific enhancer may turn off the expression of a gene critical for early development, while other variants in the same region may increase enhancer activity, and still other variants may not have an effect on transcript expression.

Substantial progress has been made in the experimental profiling and integrative analysis of epigenomic marks, such as histone marks and DNA accessibility, across a wide range of tissues and cell types. Histone marks are commonly used to identify regulatory elements; for example, H3K4me3 for active promoter regions and H3K27ac/H3K4me1 for active enhancer regions. Moreover, histone marks and chromatin accessibility can be integrated with chromatin states. These works have been instrumental to annotating the genome with tissue-specific regulatory elements.

At the same time, deep learning sequence modeling techniques have been successfully applied to learn sequence features that are predictive of transcription factor binding and histone modifications. These models are powerful tools for inferring the mutational outcome of transcription factor binding and histone modifications, such as whether a variant causes an increase or decrease of GATA2 binding. However, these predictions each focus only a very specific aspect of sequence properties, and we still lack an integrated, global view on sequence regulatory activities, limiting the interpretability and effectiveness of sequence-based analysis of genomic variants and human genetics data.

Here, a global map is described that details sequence regulatory activity. In some embodiment a deep learning sequence model-based framework predicts a comprehensive compendium of chromatin profiles for any sequence or variant. In some embodiments, the framework then maps the sequence to regulatory activities quantitatively with a novel vocabulary of sequence classes. Sequence classes are descriptors of types of regulatory activity that can be attributed to a sequence and cover diverse types of regulatory activities across the whole genome by integrating sequence-based predictions from diverse histone marks, transcription factors, and chromatin accessibility across cell types. In some embodiments, sequence classes can be used to classify and quantify any sequence based on the deep learning model framework. Therefore, sequence classes allow for quantitatively mapping any mutation to its impact on broad and cell type-specific regulatory activities.

This framework can integrate a broad set of predictions of transcription factor binding and histone modifications, which are referred to as first-order effects. These predictions can be utilized to model regulatory activities such as promoter or cell type-specific enhancer activity, which are referred to as second-order effects. For example, a variant might affect the binding of Pou5F1, Sox2, and Nanog, and the combination of these first-order effects can predict a second-order effect such as the disruption of embryonic stem cell-specific enhancer activity. In some embodiments, a set of sequence classes are used to systematically classify and quantify all major predictable second-order effect categories across the genome. This framework would not only allow a user to directly map any sequence to its first-order effects, but also provide a comprehensive, global view of regulation that incorporates all second-order regulatory activities predictions.

This framework thus allows an interpretable and systematic integration of sequence-based regulatory activity predictions (intrinsic information, based on sequence function) with human genetics data (extrinsic information, based on variant-phenotype association) for discovering the regulatory basis of human traits and disease. Moreover, variant effect prediction at the sequence-class-level newly enables the interpretation of regulatory mechanisms for disease mutations and can differentiate between gain-of-function and loss-of-function regulatory mutations. The regulatory and tissue-specific view provided by sequence classes suggests new mechanisms for individual disease-associated variants; for example, for mutations in blood-related diseases with unknown mechanisms, these mutations were linked to the malfunctioning of cell-type-specific enhancers using sequence classes.

### Overview of Effects of Variants on Transcription Regulation

In this disclosure is described various methods and processes for interpreting sequence data and the effect of sequence on first-order cis-regulatory targets and the further second-order effect on regulatory activities, which can impact the transcription of one or more genes. These various methods and processes, in accordance with the various embodiments, further interpret the effect of variants within a sequence on the first-order cis-regulatory targets and the second-order effect on regulatory activities. To perform these methods and processes, a deep-learning computational model can be utilized that is trained to predict these effects.

To predict first order cis-regulatory targets, in accordance with various embodiments, a computational model can include one or more of: a convolutional network, residual dilated convolution layers, and a spatial basis function layer. Cis-regulatory data can be utilized to train these aspects of the computational model. Cis-regulatory data can include (but is not limited to) DNA-binding protein factor profiles, histone mark profiles, and/or chromatin accessibility profiles. In numerous embodiments, a trained model will predict the cis-regulatory factors for a particular sequence. In some embodiments, a trained model will predict the DNA-binding protein factor profiles, histone mark profiles, and/or chromatin accessibility profiles for a particular sequence.

To predict second order effect on regulatory activities, in accordance with various embodiments, a dimensionality reduction and clustering is performed on the predicted cis-regulatory target results. Performing dimensionality reduction and clustering can produce clusters of sequences with similar regulatory activity, and in some embodiments, these clusters can be assigned into a sequence class. Sequence classes can be defined by their regulatory activity, including (but are not limited to) promoter classes, enhancer classes, CTCF cohesin classes, polycomb classes, heterochromatin classes, and transcription classes. In some embodiments, sequence classes are defined by the histone marks and DNA-binding proteins associated with the sequences in each cluster. For example, the active promoter histone mark H3K4me3 can be utilized to define one or more promoter classes; the enhancer histone marks H3K4me1 and H3K27ac can be utilized to define one or more enhancer classes; the polycomb-repressed region mark H3K27me3 can be utilized to define one or more polycomb classes; the heterochromatin mark H3K9me3 can be utilized to define one or more heterochromatin classes; transcription elongation marks H3K36me3 or H3K79me2 can be utilized to define one or more transcription classes; and CTCF and cohesin marks can be utilized to define CTCF cohesin classes.

Performing dimensionality reduction and clustering can also provide a vector score for each sequence within the sequence class. Thus, each sequence can be quantitatively assessed for its ability to provide its function. Higher scores indicate that the sequence provides higher regulatory activity for a given sequence class. For example, a sequence within the enhancer class with a higher score is predicted to have stronger enhancer activity on the one or more genes that the enhancer regulates. Likewise, a sequence within the polycomb class with a higher score is predicted to have stronger repressor activity on the one or more genes that the polycomb sequence regulates. Furthermore, variants within a sequence can be assessed to determine the effect of the variant on regulatory activity, as compared to another sequence (e.g., a reference sequence or alternative variant sequence). For example, if a variant within an enhancer sequence produces a higher score than the enhancer sequence without the variant, then it is predicted that the sequence with the variant provides greater enhancer activity. Likewise, if a variant within an enhancer sequence produces a lower score than the enhancer sequence without the variant, then it is predicted that the sequence with the variant provides less enhancer activity.

In several embodiments, results of processes to determine trait regulatory activity of sequences and/or variants are utilized in various downstream applications, including (but not limited to) experimental analysis of the sequence and/or variant, diagnosis of an individual, treatment of an individual and/or development of diagnostic assays. These embodiments are described in greater detail in subsequent sections.

### Processes to Yield Cis-Regulatory Effects of a Genetic Sequence

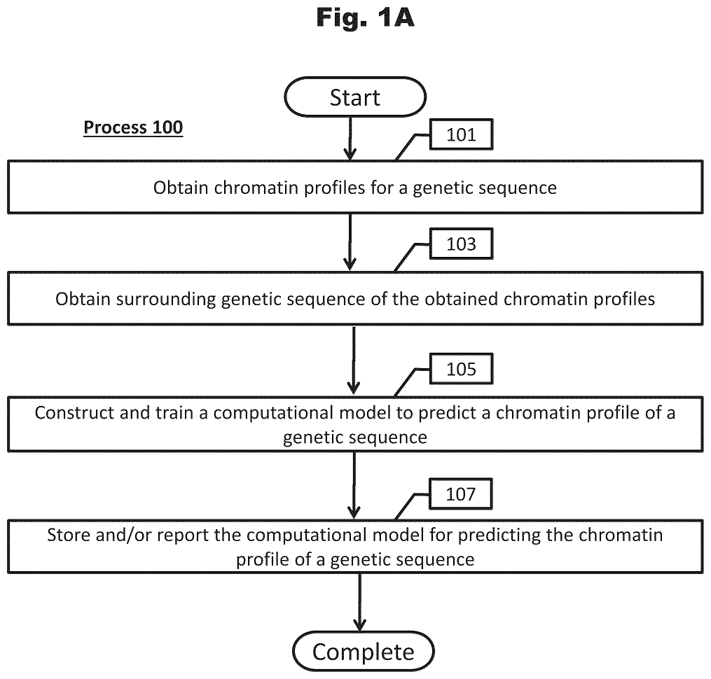

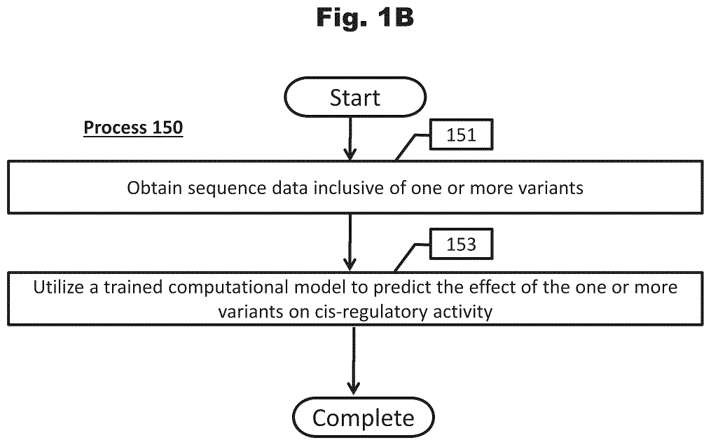

Several embodiments are directed to training a computational model to predict a cis-regulatory effect. In many embodiments, a computational model is trained utilizing chromatin profiles and sequence information. A conceptual illustration of a process to construct and train a computational model to predict a cis-regulatory effect of a genetic sequence is provided in FIG. 1A. The process can begin by obtaining (101) chromatin profiles for a genetic sequence. The genetic sequence to be analyzed is any genetic sequence in which chromatin profile data has been generated. In some embodiments, the genetic sequence is a whole or partial genome of an organism. In some embodiments, the organism is human. In various embodiments, a human reference genome is utilized, such as (for example) the GRCh38/hg38, the GRCh37/hg19, the NCBI36/hg18, the NCBI35/hg17, the NCBI34/hg16, or any combination thereof.

Chromatin profile data are any data that provide an indication of transcriptional regulatory activity associated with DNA and chromatin. Chromatin profile data include (but are not limited to) DNA-binding protein factor profiles, histone mark profiles, and/or chromatin accessibility profiles. Chromatin profile data for the human genome can be acquired by performing experimentation and/or from various databases that maintain chromatin regulatory data. Databases that can be utilized to acquire cis-regulatory data include (but are not limited to) the Cistrome Project (www.cistrome.org), the ENCODE: Encyclopedia of DNA Elements Project (www.encodeproject.org), and the Roadmap Epigenomics Project (www.roadmapepigenomics.org). The higher number of chromatin profiles can improve prediction of a computational model. Accordingly, in various embodiments, at least 1000 chromatin profiles are obtained, at least 5000 chromatin profiles are obtained, at least 10,000 chromatin profiles are obtained, at least 15,000 chromatin profiles are obtained, or at least 20,000 chromatin profiles are obtained. Further, greater breadth and variety of cell-lines and tissue-types represented by the chromatin profiles can improve prediction of a computational model. Accordingly, in various embodiments, the chromatin profiles are generated from at least 100 cell lines and tissues, at least 250 cell lines and tissues, at least 500 cell lines and tissues, or at least 1000 cell lines and tissues.

Methods to generate chromatin profiles are well known in the art. Generally, chromatin profiles can be determined utilizing various epigenetic assays including (but not limited to) chromatin immunoprecipitation sequencing (ChIP-seq), DNase I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), and methyl array.

Process 100 further obtains (103) genetic sequence in association with chromatin profiles and sequences lacking association with chromatin profiles. Accordingly, the genetic sequences are sampled across a whole or partial genome and associated with chromatin profile data (or lack thereof). When the center region of a specific genetic sequence has a particular chromatin peak greater than a set threshold, that genetic sequence is positively labeled with that chromatin peak. And when the center region of a specific genetic sequence has a no particular chromatin peaks greater than a set threshold, that genetic sequence is negatively labeled as having no peak. Genetic samples with greater sequence length provide more sequence-based data to train a computational model, however, extended sequence lengths increase computation time and effort. In some embodiments, the sequence length of a genetic sample is between 100 bp and 10,000 bp. In various embodiments, the sequence length of a genetic sample is about 100 bp, about 200 bp, about 500 bp, about 1000 bp, about 2000 bp, about 3000 bp, about 4000 bp, about 5000 bp, or about 10,000 bp. In reference to sequence length of a sample, the term “about” refers to plus or minus 10%, 20%, 30%, 40%, or 50%. In some embodiments, the chromatin profile peak is centered in the genetic sample such that an equal amount of flanking sequence is upstream and downstream the peak. In some embodiments, the chromatin profile peak is askew in the genetic sample such that the amount of sequence upstream the peak is unequal to the amount of sequence downstream.

Utilizing the obtained chromatin profiles and genetic sequence samples, a computational model is constructed and trained (105) to predict a chromatin profile of a genetic sequence. Any appropriate computational model can be utilized capable of predicting a chromatin profile based on genetic sequence. In several embodiments, the computational model is a deep neural network. In some embodiments, the computational model is a convolutional neural network, a recurrent neural network, a transformer neural network, or any combination thereof. In some embodiments, the computational model is composed of one or more components that can act concurrently or sequentially. In particular embodiments, a computational model has an architecture of three sequential components: 1) a convolutional network with dual linear and nonlinear paths, 2) residual dilated convolution layers, 3) a spatial basis function transformation layer and an output layer. It has been found that this three-component architecture has provided at least 18% improvement in computation over previously generated models (for more description of previous models, see J. Zhou, et al., bioRxiv 319681 (May 11, 2018), the disclosure of which is incorporated herein by reference).

A constructed and trained model can predict the chromatin profile of a genetic sequence. Accordingly, any genetic sequence compatible with the model can be assessed by the model to predict the cis-regulatory effects on that sequence based on the sequence information alone. The constructed and trained computational model for predicting the chromatin profile of a genetic sequence can be stored and/or reported 107. In some embodiments, the computational model may be used in many further downstream applications, including (but not limited to) predicting transcriptional regulatory activity for one or more genes.

While a specific example of a process for predicting chromatin profiles from genetic sequence data is described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

Several embodiments are directed towards determining the effect of one or more variants on cis-regulatory activity. Provided in FIG. 1B is a conceptual illustration of a process for predicting the effect of one or more variants on the cis-regulatory activity for a particular genetic sequence. The process generally utilizes a genetic sequence with variants and enters the sequence into a trained computational model that predicts a chromatin profile for the sequence. An example of a computational model for predicting a chromatin profile is provided in FIG. 1A.

Process 150 can begin by obtaining (151) sequence data inclusive of one or more variants. The sequence data is any genetic sequence compatible with a trained computational model (e.g., human sequence data can be utilized within a model that has been trained utilizing human genetic sequences).

A trained computational model is utilized (153) to predict the effect of the one or more variants on cis-regulatory activity. Any trained computational model capable of predicting cis-regulatory activity (e.g., chromatin profiles) can be utilized, such as (for example) the computational model described in reference to FIG. 1A. In some embodiments, the computational model is a convolutional neural network. In some embodiments, the computational model is composed of one or more components that can act concurrently or sequentially. In particular embodiments, a computational model has an architecture of three sequential components: 1) a convolutional network with dual linear and nonlinear paths, 2) residual dilated convolution layers, 3) a spatial basis function transformation layer and an output layer.

To understand the consequence of the one or more variants, the cis-regulatory activity of the sequence with variants can be compared to the cis-regulatory activity of the sequence without the variants, which may be a reference sequence or a sequence with an alternative variant. In some embodiments, the sequence with variants has the same (or near the same) sequence flanking the variant as the reference sequence. Furthermore, the cis-regulatory activity results may be utilized in downstream applications, such as assessing transcriptional regulatory activity for one or more genes, which may inform how variants within regulatory regions affect transcription levels of the genes that are regulated by the region containing the variants.

### Processes to Yield Transcriptional Regulatory Assessment

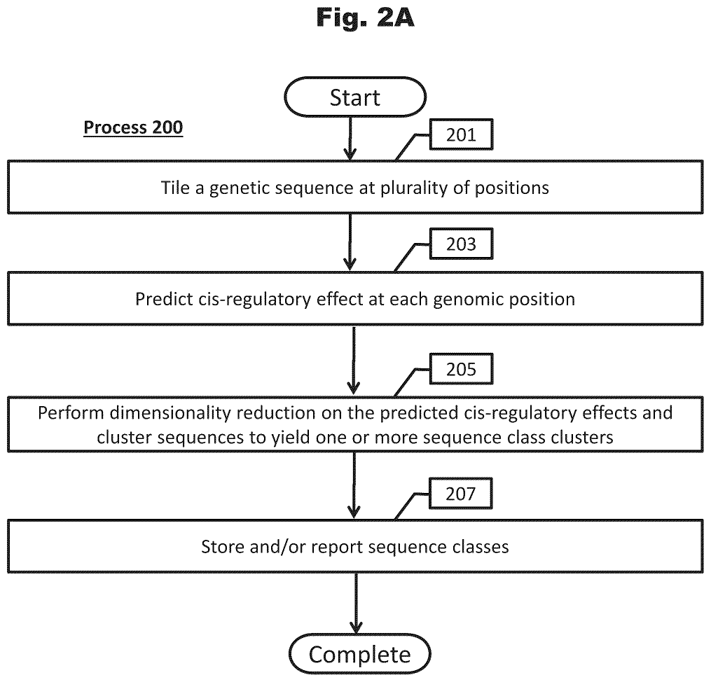

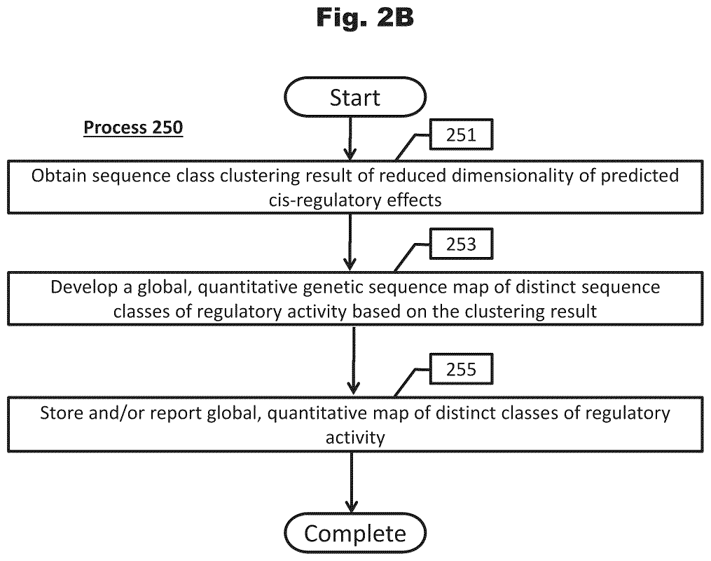

Depicted in FIG. 2A is a conceptual illustration of a process to yield one or more “sequence class” clusters via dimensionality reduction and clustering. The process utilizes cis-regulatory predictions and clusters the predictions to yield clusters of sequences with similar transcriptional regulatory behavior. The clusters of sequences with similar regulatory behavior are referred to herein as “sequence classes.”

Process 200 can begin by tiling (201) a genetic sequence at a plurality of positions. The genetic sequence to be analyzed is any genetic sequence in which chromatin profile data has been generated. In some embodiments, the genetic sequence is the whole or partial genome of an organism. In some embodiments, the organism is human. In various embodiments, a whole or partial human reference genome is utilized, such as (for example) the GRCh38/hg38, the GRCh37/hg19, the NCBI36/hg18, the NCBI35/hg17, the NCBI34/hg16, or any combination thereof.

The tiling of the genetic sequence can be performed in a step-wise fashion. In some embodiments, the sequence window of each tile is the same sequence length. In some embodiments, the sequence window length is between 100 bp and 10,000 bp. In various embodiments, the sequence window length is about 100 bp, about 200 bp, about 500 bp, about 1000 bp, about 2000 bp, about 3000 bp, about 4000 bp, about 5000 bp, or about 10,000 bp. In reference to sequence window length, the term “about” refers to plus or minus 10%, 20%, 30%, 40%, or 50%. In some embodiments, a genetic sequence is uniformly tiled such that the distance between the center of each window is the same, however, tiling does not need to be performed uniformly. In some embodiments, the distance between the center of two windows is between 20 bp and 1000 bp, which can result in overlapping windows. In various embodiments, the distance between the center of two windows is about 25 bp, 50 bp, 75 bp, 100 bp, 125 bp, 150 bp, 175 bp, about 200 bp, about 500 bp, or about 1000 bp. In reference to the distance between the center of two windows, the term “about” refers to plus or minus 10%, 20%, 30%, 40%, or 50%.

Process 200 further predicts (203) the cis regulatory effect for each sequence window tile. In some embodiments, a predicted cis regulatory effect is a predicted chromatin profile, which can be predicted by any appropriate method. In some embodiments, a chromatin profile is predicted by the process illustrated in FIG. 1A.

Process 200 also performs (205) dimensionality reduction on the predicted cis-regulatory effects and clusters sequences to yield one or more sequence class clusters. Any appropriate dimensionality reduction technique can be utilized, including (but not limited to) principal component analysis (PCA), non-negative matrix factorization (NMF), kernel PCA, graph-based kernel PCA, linear discriminant analysis (LDA), generalized discriminant analysis (GDA), T-distributed stochastic neighbor embedding (t-SNE), and uniform manifold approximation and projection (UMAP). In some embodiments, the dimensionality reduction technique is nonlinear. In some embodiments, the dimensionality reduction technique constructs a nearest neighbor graph. In some embodiments, the neighbors of the nearest neighbor graph is connected by Euclidean distance.

In some embodiments, the results of the dimensionality reduction technique are clustered by an appropriate method. Clustering methods include (but are not limited to) connectivity-based clustering, centroid-based clustering, distribution-based clustering, density-based clustering, grid-based clustering, k-means clustering, and the Louvain method for community detection. One or more resulting clusters are referred to as sequence classes. In some embodiments, a subset of the resulting clusters is utilized as sequence classes. In some embodiments, a subset of largest resulting clusters is utilized as sequence classes.

In some embodiments, the sequence classes are characterized by their enrichment of histone marks or DNA binding proteins. For example, the active promoter histone mark H3K4me3 can be utilized to define one or more promoter classes; the enhancer histone marks H3K4me1 and H3K27ac can be utilized to define one or more enhancer classes; the polycomb-repressed region mark H3K27me3 H3K27ac can be utilized to define one or more polycomb classes; the heterochromatin mark H3K9me3 can be utilized to define one or more heterochromatin classes; transcription elongation marks H3K36me3 or H3K79me2 can be utilized to define one or more transcription classes; and CTCF and cohesin marks can be utilized to define CTCF cohesin classes.

Process 200 also stores and/or reports (207) the sequence classes. In a number of embodiments, the sequence classes are used in a number of downstream applications, including (but not limited to) developing a genomic map, quantifying sequence regulatory activity, assessing variant effect on regulatory activity, and developing and/or performing diagnostics.

While a specific example of a process for yielding one or more sequence class clusters is described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

Depicted in FIG. 2B is a conceptual illustration of a process to develop a global and quantitative map of defined sequence classes of regulatory activity. This process utilizes a clustering technique to classify sequences into a particular class and quantify their regulatory activity.

Process 250 can begin by obtaining (251) a sequence class clustering result of reduced dimensionality of predicted cis-regulatory effect. In some embodiments, a predicted cis regulatory effect is a predicted chromatin profile, which can be predicted by any appropriate method. In some embodiments, a chromatin profile is predicted by the process illustrated in FIG. 1A. The dimensionality reduction and clustering of cis regulatory effects can be performed by any appropriate method. In some embodiments, the dimensionality reduction and clustering of cis regulatory effects is performed by the process illustrated in FIG. 2A.

Process 250 also develops (253) a global, quantitative genetic sequence map of distinct sequence classes of regulatory activity based on the clustering result. Accordingly, in some embodiments, each sequence within a sequence class has a sequence class score that predicts the regulatory activity for any sequence and quantifies the regulatory activity of the sequence. Sequence class scores can summarize predictions for all chromatin profiles assessed, based on weights specific to each sequence class. In some embodiments, the weights are computed by projecting predictions onto unit-length vectors that point to the center of each sequence class. Sequences that score highly for a particular sequence class have high predictions for the chromatin profiles associated with that class.

In some embodiments, a vector is computed for a sequence class i using the equation

$${v}_{i} = \frac{\overline{{p}_{s ∈ Sequence class i}}}{\overline{{‖ {p}_{s ∈ Sequence class i} ‖}_{2}}} ,$$

where pₛ represents the dimensional cis-regulatory effect prediction for sequence s. Each prediction can then be projected onto any sequence class vector to obtain a sequence class-level representation of the prediction, which is referred to herein as the sequence class score or score_{s,i}=pₛ·vᵢ^{T}.

Process 250 also stores and/or reports (255) the global, quantitative map of distinct classes of regulatory activity and/or sequence class scores. In a number of embodiments, the quantitative map and/or scores are used in a number of downstream applications, including (but not limited to) quantifying sequence regulatory activity, assessing variant effect on regulatory activity, and developing and/or performing diagnostics.

While a specific example of a process for developing a global, quantitative genetic sequence map is described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

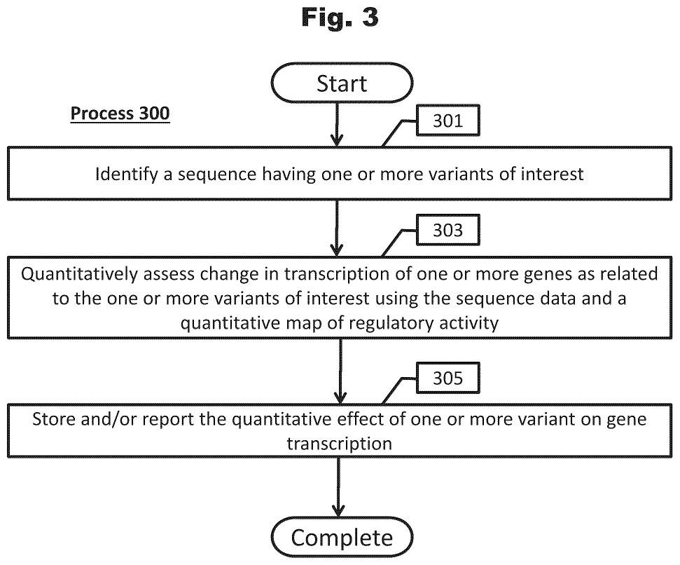

Depicted in FIG. 3 is a conceptual illustration of a process to quantitatively assess change in transcription of one or more genes associated with sequences having one or more variants using the sequence data and a quantitative map of regulatory activity. This process utilizes a clustering technique to quantify regulatory activity of sequences and further compares sequences having variants with another sequence not having the variants. This process assesses variants and their effect upon transcription levels.

Process 300 can begin by identifying (301) a sequence having one or more variants of interest. The sequence and variants of interest can be any sequence and any variants compatible with a computational model to predict cis-regulatory effects and sequence class clustering of reduced dimensionality of predicted cis-regulatory effects. In some embodiments, the sequence with variants is between 100 bp and 10,000 bp. In various embodiments, the sequence with variants is about 100 bp, about 200 bp, about 500 bp, about 1000 bp, about 2000 bp, about 3000 bp, about 4000 bp, about 5000 bp, or about 10,000 bp. In reference to sequence window length, the term “about” refers to plus or minus 10%, 20%, 30%, 40%, or 50%.

Variants are any variations in nucleotide sequence and include (but are not limited to) single nucleotide variants or polymorphisms (SNVs or SNPs), insertions, and deletions. A variant can be a variation in sequences as compared to a reference genome. In various embodiments, a variant is a variation as compared to a human reference genome, such as (for example) the GRCh38/hg38, the GRCh37/hg19, the NCBI36/hg18, the NCBI35/hg17, the NCBI34/hg16, or any combination thereof. In some embodiments, a variant is a variation as compared to another sequence in a population. For instance, a human individual's sequence can be compared with another individual's sequence or a sequence compiled from a plurality of individuals. In some embodiments, a variant is a variation as compared to another sequence within an individual. For instance, it is known that various tissues and/or individual cells of a human individual can have unique variants (especially de novo variants) and thus intra individual comparisons can be made between tissues and/or cells. In some embodiments, a variant to be analyzed is a contrived variant, which may be useful to determine the effect of novel variants not yet identified in nature. Contrived variants can be contrived by any appropriate mechanism, including (but not limited to) human contrivance or computational contrivance (e.g., random sampling or systematic sampling by computational methods).

In some embodiments, a sequence with variants is derived from a natural source. For instance, human genetic sequences can be assessed. As is understood in the art, each human individual has a unique genetic sequence that contains a unique set of variants distinct from the population. In some embodiments, a variant to be assessed is an inherited variant (i.e., derived from mother or father). In some embodiments, a variant to be assessed is a de novo variant (i.e., arise uniquely in the individual and not from the mother or father). In some embodiments, a variant to be assessed is associated with a health disorder, such as (for example) a variant derived from nucleotide sequences of a neoplastic growth or cancer. In some embodiments, a variant to be assessed has no association with phenotype etiology or health disorder etiology. In some embodiments, a variant to be assessed is a common variant (minor allele frequency greater than 5%). In some embodiments, a variant to be assessed is a rare variant (minor allele frequency less than 5%). In various embodiments, a variant to be assessed has a minor allele frequency greater than 10%, less than 10%, less than 5%, less than 1%, less than 0.5%, less than 0.1%, less than 0.01%, or less than 0.001%.

Process 300 also quantitatively assesses (303) change in transcription of one or more genes as related to the one or more variants of interest using the sequence data and a quantitative map of regulatory activity. In order to quantitatively assess changes in transcription, in accordance with some embodiments, a sequence with one or more variants of interest is assessed utilizing a computational model to predict the cis-regulatory effects of the sequence. Any appropriate method to predict the cis-regulatory effects of a sequence with one or more variants can be utilized. In some embodiments, the process portrayed in FIG. 1B is utilized to predict the cis-regulatory effects of a sequence with one or more variants.

In some embodiments, dimensionality reduction is performed on the predicted cis-regulatory effect of a sequence with variants and then clustered into a sequence class. Any appropriate method to perform dimensionality reduction and clustering of a sequence with one or more variants can be utilized. In some embodiments, the process portrayed in FIG. 2A is utilized to perform dimensionality reduction and clustering of a sequence with one or more variants.

The result of performing dimensionality reduction and clustering into a sequence class will yield a sequence class score for the sequence with one or more variants. In some embodiments, a vector is computed for a sequence class i using the equation

$${v}_{i} = \frac{\overline{{p}_{s ∈ Sequence class i}}}{\overline{{‖ {p}_{s ∈ Sequence class i} ‖}_{2}}} ,$$

where pₛ represents the cis-regulatory effect prediction for sequence s. Each prediction can then be projected onto any sequence class vector to obtain a sequence class-level representation of the prediction, which is referred to herein as the sequence class score or score_{s,i}=pₛ·vᵢ^{T}.

In some embodiments, predicted sequence-class-level variant effects are represented by the difference between the sequence class scores of the sequences carrying the reference allele and the alternative allele, or score_{v,i}=score_{alt,i}−score_{ref,i}, quantitatively capturing the change of transcription of one or more genes as related to the one or more variants of interest. Further, in some embodiments, the predicted variant effects on histone marks are normalized for nucleosome occupancy. To perform normalization of predicted variant effects, the sum of all histone profile predictions can be used as an approximation of nucleosome occupancy and all histone mark predictions can be adjusted to remove the impact of nucleosome occupancy change:

$${p}^{hm *} = {p}_{ref}^{hm} \frac{{\sum }_{k} {p}_{ref}^{{hm}^{k}} + {\sum }_{k} {p}_{alt}^{{hm}^{k}}}{{\sum }_{k} {p}_{ref}^{{hm}^{k}}} ; {p}^{hm *} = {p}_{alt}^{hm} \frac{{\sum }_{k} {p}_{ref}^{{hm}^{k}} + {\sum }_{k} {p}_{alt}^{{hm}^{k}}}{{\sum }_{k} {p}_{alt}^{{hm}^{k}}}$$

where

$${\sum }_{k} {pred}_{ref}^{{hm}^{k}}$$

represents the sum over all histone mark predictions (among 21907-dimensions of a prediction) for the reference allele.

Process 300 also stores and/or reports (305) the quantitative effect of one or more variants on gene transcription. In a number of embodiments, the quantitative effect and/or scores are used in a number of downstream applications, including (but not limited to) assessing variant effect on regulatory activity, and developing and/or performing diagnostics.

While a specific example of a process for quantitatively assessing changes in transcription related to one or more variants is described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications.

### Biochemical Analysis of Transcriptional Regulation

A number of embodiments are directed towards biochemical assays to be performed based on the results of sequences and/or variants identified to affect transcriptional regulation. Accordingly, in some embodiments, computational methods are performed to determine transcriptional regulatory effects of sequences and/or variants and based on those determinations a biochemical assay is performed to assess the computational results. In various embodiments, transcriptional regulatory effects of sequences and/or variants and/or their pathogenicity is determined by performing one or more of the computational processes described in FIGS. 1A, 1B, 2A, 2B, and/or 3. It should be noted, however, that any method capable of determining transcriptional regulatory effects of sequences and/or variants can be utilized within various embodiments.

In many embodiments, biochemical methods are performed as follows:
a) identify a sequence and/or a variant of interest
b) computationally determine transcriptional regulatory effects of the sequence and/or variant of interest
c) based on regulatory effects of the sequence and/or variants, perform a biochemical assay to assess transcription and/or cell function.

A number of biochemical assays can be performed on the basis of a computational determination of a sequence's and/or a variant's transcriptional regulatory effect. Generally, biochemical assays will provide a confirmatory assessment of sequence and/or variant effect on various biological functions, which include effects on chromatin formation, chromatin binding, associated gene transcription, cellular function, and disorder pathology. A number of biochemical assays are known in the art to assess a sequence's and/or a variant's effect, including (but not limited to) chromatin immunoprecipitation sequencing (ChIP-seq), DNAse I hypersensitivity sequencing (DNase-seq), Assay for Transposase-Accessible Chromatin sequencing (ATAC-seq), Formaldehyde-Assisted Isolation of Regulatory Elements (FAIRE-seq), Hi-C capture sequencing, bisulfite sequencing (BS-seq), methyl array, transgene expression analysis (e.g., luciferase and eGFP), qPCR, RNA hybridization (e.g., ISH), RNA-seq, western blot, immunodetection, flow cytometry, enzyme-linked immunosorbent assay (ELISA), mass spectrometry, and cellular assays.

Many embodiments are directed toward generating a nucleic acid molecule. In some embodiments, a nucleic acid having the sequence and/or the variant of interest is synthesized via phosphoramidite chemistry. In some embodiments, a nucleic acid having the sequence and/or the variant of interest is generated via polymerase chain reaction, ligation, recombination, or other molecular technique known in the art. In some embodiments, a nucleic acid having the sequence and/or the variant of interest is incorporated into a plasmid construct.

Several embodiments are also directed towards manipulating genetic material in order to analyze sequences and/or variants. In some embodiments, a particular sequence and/or a particular variant is incorporated into an expression construct for analysis. The expression construct can be transfected into a cell to express the construct and then biochemical assays can be performed on the transfected cell. In some embodiments, a particular sequence and/or a particular variant is incorporated into at least one allele of the DNA of a biological cell to be assessed in cell culture and/or within an animal model. Several methods are well known to introduce sequence and/or variant mutations within an allele, including (but not limited to) CRISPR mutagenesis, Zinc-finger mutagenesis, and TALEN mutagenesis. In some embodiments, a common variant is changed into a rare variant. In some embodiments, a rare variant is changed into a common variant. In some embodiments, a sequence and/or a variant that is introduced into a biological cell is naturally occurring. In some embodiments, a sequence and/or a variant that is introduced into a biological cell is contrived.

Various embodiments are directed towards development of cell lines and/or animal models having a particular set of one or more sequences and/or variants. In some embodiments, a cell line or animal model can be manipulated by genetic engineering to harbor a set of one or more sequences and/or variants. In some embodiments, a cell line can be derived from an individual (e.g, from a biopsy) which would harbor the one or more sequences and/or variants identified in that individual. In some embodiments, a cell line from an individual can be genetically manipulated to “correct” a set of rare sequences and/or variants. In some embodiments, a cell line or animal model is manipulated to introduce a set of one or more sequences and/or variants that is identified in that individual. In some instances, the individual being assessed has a particular medical disorder or phenotype of interest and assessment of the set of one or more sequences and/or variants determines which, if any, of the sequences and/or variants are associated with the medical disorder or phenotype.

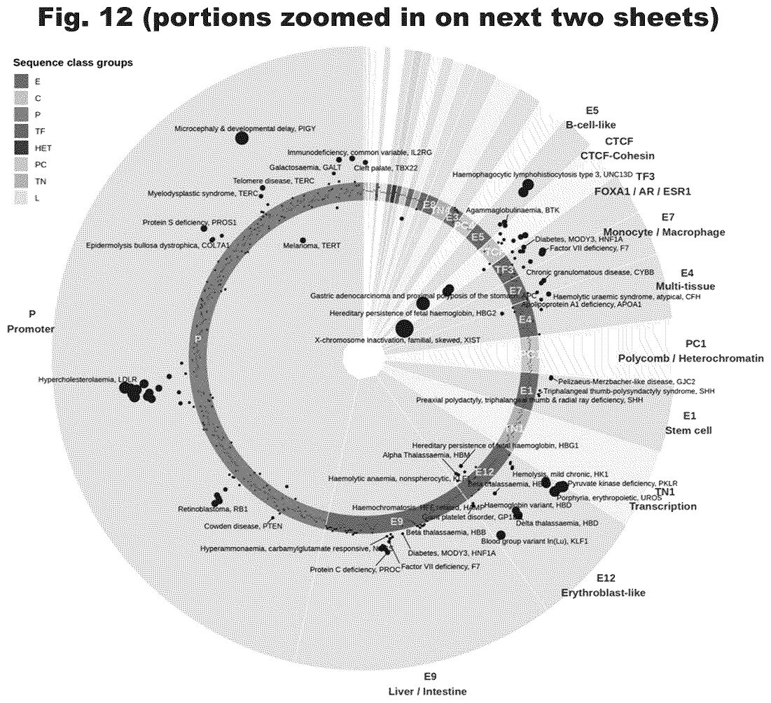

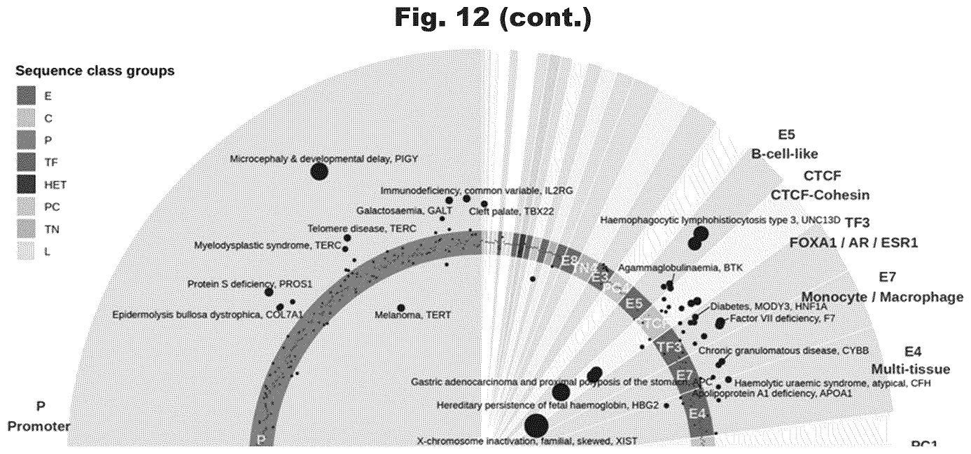

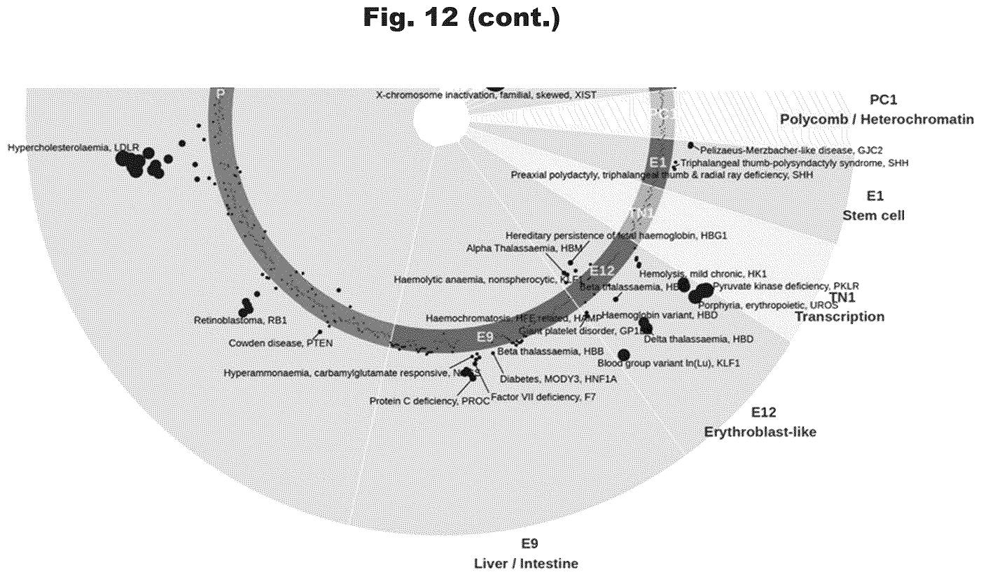

A particular sequence and/or variant can influence transcription differently in various tissues and cell types (see FIG. 12 and corresponding description for particular examples). Further, a computational framework that is trained utilizing chromatin profiles with an expansive variety of cell types and tissues can predict the regulatory activity with cell line and/or tissue specificity. For example, the computational framework described in the exemplary data utilizes 21,907 chromatin profiles from over 1300 cell lines and tissues and thus allows for delineation of regulatory activity based on cell line and tissue type. Biochemical assays can be performed in particular cell lines and/or tissues to assess the transcriptional regulatory activity in those cell lines and/or tissues. In some embodiments, a sequence and/or variant is computationally predicted to have an effect in one or more particular cell lines and/or tissues but have little to no effect in other cell lines and/or tissues. Further, biochemical analysis can be performed in particular cell lines and/or tissues to assess whether particular sequences and/or variants have particular effects within one or more particular cell lines and/or tissues. In many embodiments, the cell lines and/or tissues that are assessed are human.

### Diagnostics Related to Transcriptional Regulation

Various embodiments are directed to methods of diagnosis and development of diagnostics related to transcriptional regulatory activity. Diagnostics and methods of diagnosis can be utilized in various different clinical settings. In some embodiments, a patient may have a portion or all of their genetic data sequenced, revealing variants (including, but not limited to, inherited, de novo, novel, and/or unannotated variants), which can be further assessed to predict their transcriptional regulatory effect. In some embodiments, a genetic sequencing pipeline, such as those provided by commercial sequencing companies, can identify variants in their customers, which can be further assessed to predict their transcriptional regulatory effect. Further, in some embodiments, diagnostics for a particular phenotype and/or medical disorder can be developed by collecting genetic sequence data from one or more individuals diagnosed with the particular phenotype and/or medical disorder and variants can be identified that affect transcriptional regulation, which can be utilized to develop a streamlined diagnostic.

### Methods of Diagnosis

Various embodiments are directed towards diagnosing individuals having one or more variants, including (but not limited to) inherited, de novo, novel, and/or unannotated variants. In some embodiments, a computational framework is utilized to predict the transcriptional regulatory effect of the variants. Based on the predicted transcriptional regulatory effect on one or more genes, a diagnosis can be inferred. For instance, if it is found that one or more novel and/or unannotated variants affects the transcription of one or more genes involved in maintaining healthy blood pressure, such as (for example) the CYP11B1 or CYP11B2 genes, then a molecular diagnosis can be inferred. Based on the molecular diagnosis, further clinical diagnostics and/or treatments can be performed.

An exemplary diagnostic method can be performed as follows:
a) obtain genetic sequence data of the individual to be diagnosed
b) identify one or more novel and/or unannotated variants
c) predict the regulatory effect of the one or more novel and/or unannotated variants on one or more genes
d) diagnose the individual based on the regulatory effect on the one or more genes
   Prediction of the regulatory effect of one or more variants can be performed utilizing the various computational processes described herein. Based on a diagnosis, an individual can be treated accordingly.

This diagnostic method improves upon traditional diagnostic methods, especially in cases in which the individual does not have any variant previously associated with a medical disorder. Because an individual is likely to have thousands to millions of unannotated variants, traditional genetic tests of examining a single gene, variant, and/or are not practical. As described herein, however, a computational framework can assess the regulatory effect of each variant and identify the variants with the greatest effect on gene transcription.

Various embodiments are directed towards providing a genetic sequencing pipeline utilizing a computational framework to predict the transcriptional regulatory effect of unannotated variants. In some embodiments, a service is provided that sequences the genetic sequence of an individual and interprets the sequence and variant results to provide phenotypic and medical evaluation. In some embodiments, the sequencing service can utilize predicted transcriptional regulatory effect on one or more genes to better interpret unannotated variants. Based on the interpretation of unannotated variants, an individual can be informed of their phenotypic and medical status.

An exemplary sequencing method can be performed as follows:
a) obtain genetic biomolecules of the individual to be analyzed
b) sequence the genetic biomolecules
c) identify one or more unannotated variants
d) predict the regulatory effect of the one or more unannotated variants on one or more genes
e) determine a phenotypic or a medical status of the individual based on the regulatory effect on the one or more genes
   Prediction of the regulatory effect of one or more unannotated variants can be performed utilizing the various computational processes described herein.

A sequencing pipeline as described improves upon traditional sequencing methods, especially in cases in which the individual has a high number of unannotated variants. Because an individual is likely to have thousands to millions of unannotated variants, traditional sequencing methods simply ignore these variants despite the fact that they could be important for phenotypic and/or medical evaluation. As described herein, however, a computational framework can assess the regulatory effect of each variant and identify the variants with the greatest effect on gene transcription.

Various embodiments are directed towards diagnostics that stratify patients based on predicted outcome as determined by the magnitude of predicted regulatory effect of variants related to a disorder. In some embodiments, a service is provided that sequences the genetic sequence of an individual having a particular disorder and interprets magnitude of predicted regulatory effect of variants to provide stratification. For instance, it has been discovered that the impact of somatic mutations derived from a tumor sample can be analyzed to predict survivability. When the impact of mutations are associated with poorer outcome, the individual can be administered a more aggressive treatment regimen. Alternatively, when impact of mutations are not associated with a poorer outcome, the individual can be administered a less aggressive treatment regimen, which may provide less side effects or discomfort. In some embodiments, the individual is diagnosed as having cancer and the diagnostic determines the severity of the cancer progression via an outcome (e.g., survivability). Although cancer is utilized as an example, it is to be understood any disorder that can be stratified based on an outcome. Furthermore, although survivability was utilized as an outcome in the cancer example, any outcome can be utilized. In cancer for instance, outcome can be likelihood of recurrence or likelihood of metastasis.

An exemplary diagnostic method can be performed as follows:
a) obtain genetic sequence data of the individual to be assessed
b) identify one or more variants (e.g., somatic variants of tumor)
c) predict the impact of regulatory effect of the one or more variants
d) stratify the individual based on the predicted magnitude of impact of effect of the one or more variants
   Prediction of the impact regulatory effect of one or more variants can be performed utilizing the various computational processes described herein.

A sequencing pipeline as described improves upon traditional sequencing diagnostics, especially in cases in which the individual has a high number of variants, and/or especially somatic variants of a cancer. Because the impact of variants is not readily detectable, traditional sequencing methods cannot provide a diagnostic that can stratify patients based on magnitude of impact. As described herein, however, a computational framework can assess the regulatory effect of each variant and determine magnitude of impact of these variants.

Various embodiments are directed towards determining heritability risk for complex disorders (see list of complex disorders that can be assessed below). In some embodiments, a service is provided that sequences the genetic sequence of an individual to determine heritability risk of a complex disorder. In some embodiments, the service is performed as a part of general screening. In some embodiments, the service is performed when at least one family member has or is at risk of developing a complex disorder. Combined with linkage disequilibrium score regression (LDSR) or other heritability risk calculators, the risk can be assessed by sequence class annotations in genome-wide association studies, which can identify and prioritize transcriptional regulatory regions that contribute to the heritability risk for the complex disorder.

An exemplary diagnostic method can be performed as follows:
a) obtain genetic sequence data of the individual to be diagnosed
b) identify one or more inherited variants
c) predict a magnitude of impact of regulatory effect of the one or more variants
d) combine a heritability risk calculator that uses clinical information (e.g., family history, age, sex, receptor status) with predicted mutational impacts for mutations that confer risk
   Prediction of the impact regulatory effect of one or more variants can be performed utilizing the various computational processes described herein.

A sequencing pipeline as described improves upon traditional heritability risk assessment, especially in cases in which there is a high number of variants and/or when the impact of variants associated with heritability is unknown. Because the impact of variants is not readily detectable, traditional sequencing methods cannot provide a diagnostic that provides magnitude of impact of variants associated with risk heritability. As described herein, however, a computational framework can assess the regulatory effect of each variant and determine magnitude of impact of these variants.

Various embodiments are directed towards developing diagnostic kits and/or methods for a particular phenotype and/or medical disorder utilizing a computational framework to predict the transcriptional regulatory effect of the novel and/or unannotated variants that are potentially associated with the phenotype and/or medical disorder. To develop a diagnostic, one or more individuals each having a particular phenotype and/or medical disorder can have their genetic sequence data analyzed to identify novel and/or unannotated variants associated with phenotype and/or medical disorder, especially a complex medical disorder. For instance, a collection of one or more individuals on the autism spectrum disorder (ASD) can have their genetic sequence data analyzed to identify novel and/or unannotated variants associated with ASD. Further, clustering techniques can be performed on the variant effect of transcriptional regulation from the collection of individuals to identify key clusters associated with the phenotype and/or medical disorder. Based on the predicted transcriptional regulatory effect on one or more genes, diagnostic variants of importance can be inferred. A diagnostic kit and/or method can be developed that streamlines the process of assessing the important diagnostic variants. Furthermore, utilization of a diagnostic kit and/or method can provide a diagnosis such that treatments can be performed.

An exemplary method to develop a diagnostic kit and/or method can be performed as follows:
a) obtain genetic sequence data of a collection of one or more individuals each having a particular phenotype and/or medical disorder
b) identify variants from the collection of genetic sequence data
c) predict the regulatory effect of the variants on one or more genes
d) cluster the predicted regulatory effects to identify diagnostic variants of importance
e) develop a kit and/or method that streamlines the assessment of diagnostic variants of importance
   Prediction of the regulatory effect of one or more variants can be performed utilizing the various computational processes described herein. Diagnostic kits and/or methods can be developed by various molecular techniques, including (but not limited to) sequencing assays and in situ hybridization assays.

Furthermore, various embodiments are directed to diagnostic kits and/or methods for diagnosing complex (i.e., multifactorial) disorders, including (but not limited to) autism spectrum disorder, Alzheimer disease, arthritis, asthma, bipolar disorder, cancer, cleft lip and/or palate, coronary artery disease, Crohn's disease, dementia, depression, diabetes (type II), heart disease, heart failure, high cholesterol, hypertension, hypothyroidism, irritable bowel syndrome, obesity, osteoporosis, Parkinson disease, rhinitis (allergic and nonallergic), psoriasis, multiple sclerosis, schizophrenia, sleep apnea, spina bifida, and stroke.

### Diagnostic Kits

Embodiments are directed towards genomic loci sequencing and/or single nucleotide polymorphism (SNP) array kits to be utilized within various methods as described herein. As described, various methods can diagnose an individual for a complex trait by examining diagnostic variants in various regulatory genomic loci. Accordingly, a number of embodiments are directed towards genomic loci sequencing and SNP array kits that cover a set of genomic loci to diagnose a particular trait. In some instances, the set of genomic loci are identified by a computational model, such as those described herein.

A number of targeted gene sequencing protocols are known in the art, including (but not limited to) partial genome sequencing, primer-directed sequencing, and capture sequencing. Generally, targeted sequencing involves selection step either by hybridization and/or amplification of the target sequences prior to sequencing. Therefore, embodiments are directed to sequencing kits that target genomic loci that are known to harbor diagnostic variants to diagnose a particular medical disorder.

Likewise, a number of SNP array protocols are known in the art. In general, chip arrays are set with oligo sequences having a particular SNP. Sample DNA derived from an individual can be processed and then applied to SNP array to determine sites of hybridization, indicating existence of a particular SNP. Thus, embodiments are directed to SNP array kits that target particular SNPs that are known to be pathogenic in order to diagnose a particular medical disorder.

The number of genomic loci and/or SNPs to include in a sequencing kit can vary, depending on the genomic loci and/or SNPs to examine for a particular trait and the computational model to be used. In some embodiments, the genomic loci and/or SNPs to be examined are identified by a computational model, such as the computational models described herein. In various embodiments, the number of genomic loci in a sequencing kit are approximately, 100, 1000, 5000, 10000, 20000, 30000, 40000, 50000, 60000, 70000, 80000, 90000, 100000, 150000, or 200000 loci. In various embodiments, the number of SNPs in an array kit are approximately, 1000, 10000, 50000, 100000, 200000, 300000, 400000, 500000, 600000, 700000, 800000, 900000, 1000000, 1500000, or 2000000 SNPs. In some embodiments, all identified loci are included in a kit. In some embodiments, only a subset of the loci are included. It should be understood that precise number and positions of loci can vary as the classification model can be updated with new data or recreated with a different data set (especially for different traits, and/or subtypes of traits).

### Exemplary Clinical Assessments and Medications for Complex Disorders

Several embodiments are directed to the use of medications and/or dietary supplements to treat an individual based on their medical disorder diagnosis. In some embodiments, medications and/or dietary supplements are administered in a therapeutically effective amount as part of a course of treatment. As used in this context, to “treat” means to ameliorate at least one symptom of the disorder to be treated or to provide a beneficial physiological effect.

A therapeutically effective amount can be an amount sufficient to prevent, reduce, ameliorate or eliminate symptoms of disorders or pathological conditions susceptible to such treatment, such as, for example, autism, bipolar disorder, depression, schizophrenia, or other diseases that are complex. In some embodiments, a therapeutically effective amount is an amount sufficient to reduce the symptoms of a complex disorder.

Dosage, toxicity and therapeutic efficacy of the compounds can be determined, e.g., by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to other tissue and organs and, thereby, reduce side effects.

Data obtained from cell culture assays or animal studies can be used in formulating a range of dosage for use in humans. If the pharmaceutical is provided systemically, the dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration or within the local environment to be treated in a range that includes the IC₅₀ (i.e., the concentration of the test compound that achieves a half-maximal inhibition of neoplastic growth) as determined in cell culture. Such information can be used to more accurately determine useful doses in humans. Levels in plasma may be measured, for example, by liquid chromatography coupled to mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments. For example, several divided doses may be administered daily, one dose, or cyclic administration of the compounds to achieve the desired therapeutic result.

A number of medications and treatments are known for several complex disorders, especially those that arise (at least in part) due to regulatory variants. Accordingly, embodiments are directed toward treating an individual with a treatment regimen and/or medication when diagnosed with a complex disorder as described herein. Various embodiments are directed to treatments of complex (i.e., multifactorial) disorders, including (but not limited to autism spectrum disorder, Alzheimer's disease, arthritis, asthma, bipolar disorder, cancer, cleft lip and/or palate, coronary artery disease, Crohn's disease, dementia, depression, diabetes (type II), heart disease, heart failure, high cholesterol, hypertension, hypothyroidism, irritable bowel syndrome, obesity, osteoporosis, Parkinson disease, rhinitis (allergic and nonallergic), psoriasis, multiple sclerosis, schizophrenia, sleep apnea, spina bifida, and stroke.

Once diagnosed for having a risk of autism spectrum disorder, medical monitoring (e.g., regular check-ups) can be performed to look for signs of developmental delays. Various treatments include behavioral, communication, and educational therapies, each of which strive to improve a diagnosed individual's social and cognitive skills. Behavioral training, including applied behavior analysis, can be performed, in which ASD subjects are taught behavioral skills across different settings and reinforcing the desirable characteristics, such as appropriate social interactions. In some instances, speech and language pathology can be performed to improve development of language and communication skills, including the ability to articulate words wells, comprehend verbal and non-verbal clues in a range of settings, initiate conversation, develop conversational skills (e.g., appropriate time to say “good morning” or responses to questions asked). In some instances, an ASD subject is entered into special education courses. In some instances risperidone can be administered, which treats irritability often associated with ASD individuals.

Once diagnosed for having a risk of Alzheimer's disease, neurological and neuropsychological tests can be performed to check mental status. Imaging (e.g., MRI, CT, and PET) can be performed to check for abnormalities in structure or function. A number of supplements may help brain health and may be prophylactic, including (but not limited to) omega-3 fatty acids, curcumin, ginkgo, and vitamin E. Exercise, diet, and social support can help promote good cognitive health. Medications for Alzheimer's include (but are not limited to) cholinesterase inhibitors and memantine.

Once diagnosed for having a risk of arthritis, laboratory tests on various bodily fluids can be performed to determine the type of arthritis. Imaging (e.g., X-rays, CT, MRI, and ultrasound) can be utilized to detect problems in various joints. Physical therapy may help relieve some complications associated with arthritis. Medications for arthritis include (but are not limited to) analgesics, nonsteroidal anti-inflammatory drugs (NSAIDs), counterirritants, disease-modifying antiheumatic drugs, biologic response modifiers, and corticosteroids. Heat pads, ice packs, acupuncture, glucosamine, yoga, and massage are examples of various home/alternative remedies available.

Once diagnosed for having a risk of asthma, tests can be performed to determine lung function. A chest X-ray of CT scan can be performed to determine any structural abnormalities. Medications for asthma include (but are not limited to) inhaled corticosteroids, leukotriene modifiers, long-acting beta agonists, short-acting beta agonists, theophylline, and ipratropium. In some instances, allergy medications may help asthma and thus allergy shots and/or omalizumab can be administered. Regular exercise and maintaining a healthy weight may help reduce asthma symptoms.

Once diagnosed for having a risk of bipolar disorder, a psychiatric assessment can be performed to determine the feelings and behavior patterns. Psychotherapies and medications are available to treat bipolar disorder. Psychotherapies include (but not limited to) interpersonal and social rhythm therapy (IPSRT), cognitive behavioral therapy (CBT), and psychoeducation. Medications include (but not limited to) mood stabilizers, antipsychotics, antidepressants, and anti-anxiety medications. Some lifestyle changes can help manage some cycles of behavior that may worsen the condition, including (but not limited to) limiting drugs and alcohol, forming healthy relationships with positive influence, and getting regular physical activity.

Once diagnosed for having a risk of cancer, physical exams, laboratory tests and imaging (e.g., CT, MRI, PET) can be performed to determine if cancerous tissue is present. A biopsy can be extracted to confirm a growth is cancerous. Various treatments can be performed, including (but not limited to) adjuvant treatment, palliative treatment, surgery, chemotherapy, radiation therapy, immunotherapy, hormone therapy, and targeted drug therapy. Exercise and a healthy diet can help an individual mitigate cancer onset and progression.

Once diagnosed for having a risk of cleft lip or palate, ultrasound can be performed in utero to determine whether a fetus is developing a cleft lip or palate. Typical treatment is surgery to repair the cleft tissue.

Once diagnosed for having a risk of coronary artery disease, an electrocardiogram and/or echogram can be performed to determine a heart's performance. A stress test can be performed to determine the ability of the heart to respond to physical activity. A heart scan can determine whether calcium deposits. Patients having risk of coronary artery disease would benefit greatly from a few lifestyle changes, including (but not limited to) reduce tobacco use, eat healthy foods, exercise regularly, lose excess weight, and reduce stress. Various medications can also be administered, including (but not limited to) cholesterol-modifying medications, aspirin, beta blockers, calcium channel blockers, ranolazine, nitroglycerin, ACE inhibitors and angiotensin II receptor blockers. Angioplasty and coronary artery bypass can be performed when more aggressive treatment is necessary.

Once diagnosed for having a risk of Crohn's disease, a combination of tests and procedures can be performed to confirm the diagnosis, including (but not limited to) blood tests and various visual procedures such as a colonoscopy, CT scan, MRI, capsule endoscopy and balloon-assisted enteroscopy. Treatments for Crohn's disease include corticosteroids, oral 5-aminosliclates, azathioprine, mercaptopurine, infliximab, adalimumab, certolizumab pegol, methotrexate, natalizumab and vedolizumab. A special diet may help suppress some inflammation of the bowel.

Once diagnosed for having a risk of dementia, further analysis of mental function can be performed to gauge memory, language skills, ability to focus, ability to reason, and visual perception. These analyses can be performed utilizing cognitive and neuropsychological tests. Brain scan (e.g., CT, MRI, and PET) and laboratory tests can be performed to determine if physiological complications exist. Medications for dementia include cholinesterase inhibitors and memantine.

Once diagnosed for having a risk of diabetes, a number of tests can be performed to determine an individual's glucose levels and regulation, including (but not limited to) glycated hemoglobin A1C test, fasting blood sugar levels, and oral glucose tolerance test. Routine visits may be performed to get a long-term regulatory look at glucose regulation. In addition, a glucose monitor can be utilized to continuously monitor glucose levels. Diabetes can be managed by various options, including (but not limited to) healthy eating, regular exercise, medication, and insulin therapy. Medications for diabetes include (but are not limited to) metformin, sulfonylureas, meglitinides, thiazolidinediones, DPP-4 inhibitors, SGLT inhibitors, and insulin.

Once diagnosed for having a risk of heart disease, various tests can be performed to determine heart function, including (but not limited to) electrocardiogram, Holter monitoring, echocardiogram, stress test, and cardiac catheterization. Lifestyle changes can dramatically improve heart disease, including (but not limited to) limiting tobacco products, controlling blood pressure, keeping cholesterol in check, keeping blood glucose levels in a good range, physical activities, eating healthy, maintaining a healthy weight, managing stress, and coping with depression. A number of medications can be provided, as dependent on the type heart of disease.

Once diagnosed for having a risk of heart failure, various tests can be performed to confirm the diagnosis, including (but not limited to) physical exams, blood tests, chest X-rays, electrocardiogram, stress test, imaging (e.g., CT and MRI), coronary angiogram, and myocardial biopsy. Medications for heart failure include (but are not limited to) ACE inhibitors, angiotensin II receptor blockers, beta blockers, diuretics, aldosterone antagonists, inotropes, and digoxin. Surgical procedures may be necessary, and include (but are not limited to) coronary bypass surgery and heart valve repair/replacement.

Once diagnosed for having a risk of high cholesterol, blood tests can be performed to measure total cholesterol, LDL cholesterol, HDL cholesterol, and triglycerides. Medications to manage cholesterol levels include (but are not limited to) statins, bile-acid-binding resins, cholesterol absorption inhibitors, and fibrates. Supplements can also be taken, including (but not limited to) co-enzyme Q, red yeast rice extract, niacin, soluble fiber, and omega-3-fatty acids. Individuals at risk for high cholesterol should also reduce tobacco products, eat a healthy diet (avoiding saturated fat, trans fat, and salt), and get regular exercise.

Once diagnosed for having a risk of hypertension, blood pressure levels can be monitored periodically (even at home). Elevated blood pressure and hypertension benefit from lifestyle changes including, eating healthy, reducing sodium intake, regular physical activity, maintaining a proper rate, and limiting alcohol intake. Medications for hypertension include (but are not limited to) ACE inhibitors, angiotensin II receptor blockers, calcium channel blockers, alpha blockers, beta blockers, aldosterone antagonists, renin inhibitors, vasodilators, and central-acting agents.

Once diagnosed for having a risk of hypothyroidism, blood tests can be performed to measure the level of TSH and thyroid hormone thyroxine. Medications for hypothyroidism include (but are not limited to) synthetic thyroid hormone levothyroxine, which may be taken with supplements such as iron, aluminum hydroxide, and calcium to help absorption.

Once diagnosed for having a risk of irritable bowel syndrome (IBS), physical exams can be performed to confirm IBS including determining type of IBS. These exams include (but are not limited to) flexible sigmoidoscopy, colonoscopy, X-ray, and CT scan. A proper diet can be utilized to manage symptoms, including (but not limited to) high fiber fluids, plenty of fluids, and avoiding the following: high-gas foods, gluten, and FODMAPs. Medications for IBS include (but are not limited to) alosetron, eluxadoline, rifaximin, lubiprostone, linaclotide, fiber supplements, laxatives, anti-diarrheal medications, anticholinergic medications, antidepressants, and pain medications.

Once diagnosed for having a risk of obesity, a physiological test to determine body-mass index (BMI) may be performed. Obesity can be managed by various lifestyle remedies including (but not limited to) healthy diet, physical activity, and limiting tobacco products. If obesity is severe, various surgeries can be performed, including (but not limited to) gastric bypass surgery, laparoscopic adjustable gastric banding, biliopancreatic diversion with duodenal switch, and gastric sleeve.

Once diagnosed for having a risk of osteoporosis, bone density can be measured and routinely monitored using X-rays and other devices, as known in the art. Medications for osteoporosis include (but are not limited to) biphosponates, estrogen (and estrogen mimics), denosumab, and teriparatide. To reduce the risk of osteoporosis development, individuals can make various lifestyle changes, including (but not limited to) limiting tobacco use, limiting alcohol intake, and taking measures to prevent falls.

Once diagnosed for having a risk of Parkinson's disease, a single-photon emission computerized tomography (SPECT) scan can image dopamine transporter activity in the brain, which can be monitored over time. Medications for Parkinson's includes (but are not limited to) carbidopa-levodopa, dopamine agonists, MAO B inhibitors, COMT inhibitors, anticholinergics and amantadine.

Once diagnosed for having a risk of rhinitis, various tests can be performed to determine if the rhinitis is due to allergies, including (but not limited to) skin tests looking for allergic reaction, blood tests to measure responses to allergies (e.g., IgE levels). Medications for rhinitis include (but are not limited to) saline nasal sprays, corticosteroid nasal sprays, antihistamines, anticholinergic nasal sprays, and decongestants.

Once diagnosed for having a risk of psoriasis, routine physical exams of the skin, scalp and nails can be performed to look for signs of inflammation. A number of topical treatments can be performed for psoriasis, including (but not limited to) topical corticosteroid, vitamin D analogues, anthralin, topical retinoids, calcineurin inhibitors, salicylic acid, coal tar, and moisturizers. A number of phototherapies can also be performed, including (but not limited to) exposure to sunlight, UVB phototherapy, Goeckerman therapy, excimer laser, and psoralen plus ultraviolet A therapy. Medications for psoriasis include (but are not limited to) retinoids, methotrexate, cyclosporine, and biologics that reduce immune-mediated inflammation (e.g., etanercept, infliximab, adalimumab).

Once diagnosed for having a risk of multiple sclerosis (MS), various tests can be performed overtime to monitor symptoms of MS, including (but not limited to) blood tests, lumbar puncture, MRI and evoked potential tests. A number treatments can help treat acute MS symptoms and to mitigate MS progression, including (but not limited to) corticosteroids, plasma exchange, ocrelizumab, beta interferons, glatiramer acetate, dimethyl fumarate, fingolimod, teriflunomide, natalizumab, alemtuzumab, and mitoxantrone. Physical therapy and muscle relaxants also help mitigate (or prevent) MS symptoms.

Once diagnosed for having a risk of schizophrenia, a physical exam and/or psychiatric evaluation may be performed to determine if symptoms of schizophrenia are apparent. Various antipsychotics may be administered, including (but not limited to) aripiprazole, asenapine, brexpiprazole, cariprazine, clozapine, iloperidone, lurasidone, olanzapine, paliperidone, quetiapine, risperidone, and ziprasidone. Individual with risk of schizophrenia may also benefit from various psychosocial interventions, normalizing thought patterns, improving communication skills, and improving the ability to participate in daily activities.

Once diagnosed for having a risk of sleep apnea, an evaluation that monitors an individual's sleep may be performed, including (but not limited to) nocturnal polysomnography, measurements of heart rate, blood oxygen levels, airflow, and breathing patterns. Sleep apnea therapy may include the use of a continuous positive airway pressure (CPAP) device. A number of lifestyle changes have also been shown to mitigate complications associated with sleep apnea, including (but not limited to) losing excess weight, physical activity, mitigating alcohol consumption, and sleeping on side or abdomen.

Once diagnosed for having a risk of spina bifida, prenatal screening tests can be performed and routinely monitored determine if a fetus is developing spina bifida. Blood tests that can be performed include (but are not limited to) maternal serum alpha-fetoprotein test and measurement AFP levels. Routine ultrasound can be performed to screen for spina bifida. Various treatments include (but are not limited to) prenatal surgery to repair the baby's spinal cord and post-birth surgery to put the meninges back in place and close the opening of the vertebrae.

Once diagnosed for having a risk of stroke, routine monitoring can be performed to determine coronary health status, including (but not limited to) blood clotting tests, imaging (e.g., CT and MRI) to look for potential clots, carotid ultrasound, cerebral angiogram, and echocardiogram. Various procedures that can be performed include (but are not limited to) carotid endarterectomy and angioplasty. Patients having risk of stroke would benefit greatly from a few lifestyle changes, including (but not limited to) reduce of tobacco use, eat healthy foods, exercise regularly, lose excess weight, and reduce stress. Various medications can also be administered, including (but not limited to) cholesterol-modifying medications, aspirin, beta blockers, calcium channel blockers, ranolazine, nitroglycerin, ACE inhibitors and angiotensin II receptor blockers.

### Exemplary Data

Bioinformatic and biological data support the systems and methods of determining the contribution of variants on transcriptional regulatory activity and applications thereof. In the ensuing sections, exemplary computational methods and exemplary applications related to variant classifications are provided that determine the effect of the variant on transcription.

### Sequence-Based Global Map of Regulatory Activity for Deciphering Human Genetics

Sequence is at the basis of how the genome shapes chromatin organization, regulates gene expression, and impacts traits and diseases. Epigenomic profiling efforts have enabled large-scale identification of regulatory elements, yet a sequence-based map to systematically identify regulatory activities from any sequence is still lacking. A sequence-based map would be helpful for predicting the effects of any variant on these activities. This challenge is addressed with exemplary data utilizing an exemplary computational process called Sei, a new framework for integrating human genetics data with sequence information to discover the regulatory basis of traits and diseases. The framework systematically learns a vocabulary for the regulatory activities of sequences, which is referred to herein as “sequence classes,” using a new deep learning model that predicts a compendium of 21,907 chromatin profiles across >1,300 cell lines and tissues, the most comprehensive to-date, with a new deep learning sequence model. Sequence classes allow for a global view of sequence and variant effects by quantifying diverse regulatory activities, such as loss or gain of cell-type-specific enhancer function. We show that sequence class predictions are supported by experimental data, including tissue-specific gene expression, expression QTLs, and evolutionary constraints based on population allele frequencies. Finally, the framework was applied to human genetics data. Sequence classes uniquely provide a non-overlapping partitioning of GWAS heritability by tissue-specific regulatory activity categories, which we use to characterize the regulatory architecture of 47 traits and diseases from UK Biobank. Furthermore, the predicted loss or gain of sequence class activities suggest specific mechanistic hypotheses for individual regulatory pathogenic mutations. This framework is described here as an exemplary process to further elucidate the sequence basis of human health and disease.

### Developing a Comprehensive Sequence Model for 21,907 Chromatin Profiles

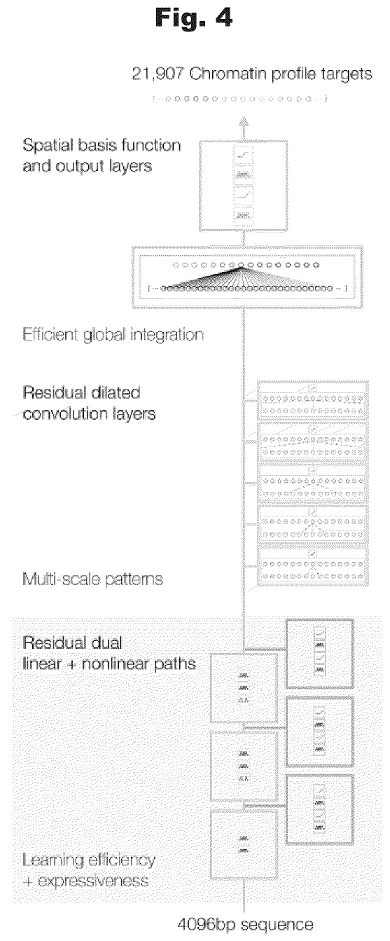

To capture the widest range of sequence features that are predictive of regulatory activities, a new deep learning sequence model was developed: the Sei model (FIG. 4), which enables the base-level interpretation of sequences by predicting 21,907 genome-wide cis-regulatory targets—including 9,471 transcription factor profiles, 10,064 histone mark profiles and 2,372 chromatin accessibility profiles—with single nucleotide sensitivity. The majority of this data (19,905 profiles) is from the Cistrome Project (R. Zheng, et al., Nucleic Acids Res. 47, D729-D735 (2019), the disclosure of which is incorporated herein by reference), a resource that uniformly processes and annotates public ChIP-, DNase-, and ATAC-seq datasets, and the remaining chromatin profiles were processed by the ENCODE (B. E. Bernstein, et al., Nature 489, 57-74 (2012), the disclosure of which is incorporated herein by reference) and Roadmap Epigenomics projects (Roadmap Epigenomics Consortium, et al. Nature 518, 317-330 (2015), the disclosure of which is incorporated herein by reference). The Sei model encompasses an estimated ˜1000 non-histone DNA-binding proteins (which we will refer to as transcription factors), 77 histone marks, and chromatin accessibility across >1300 cell lines and tissues.

To efficiently predict 21,907 chromatin profiles from sequence, a novel model architecture was designed with an improved training pipeline (FIG. 4). The Sei model uses a new residual-block architecture with a dual linear and nonlinear path design. The linear path allows for fast and statistically efficient training while the nonlinear path offers strong representation power and the capability to learn complex interactions. For scaling and performance, a layer of spatial basis functions layer was utilized, which integrates information across spatial locations with much higher memory efficiency than fully connected layers. The model takes as input a 4 kb length sequence and predicts the probabilities of 21,907 targets at the center position. The model training pipeline uses on-the-fly sampling to improve training speed and performance, which reduces overfitting by generating new training samples for every training step.

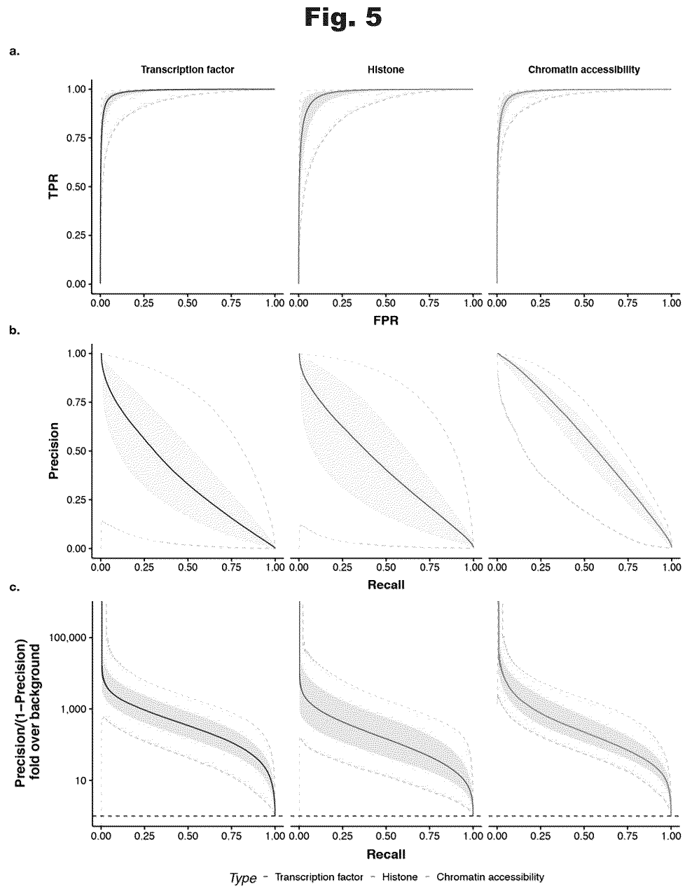

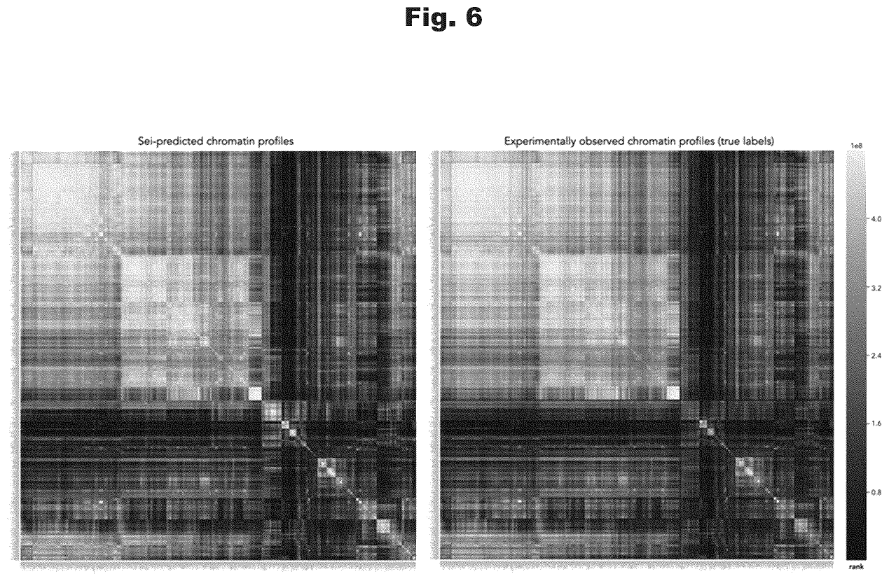

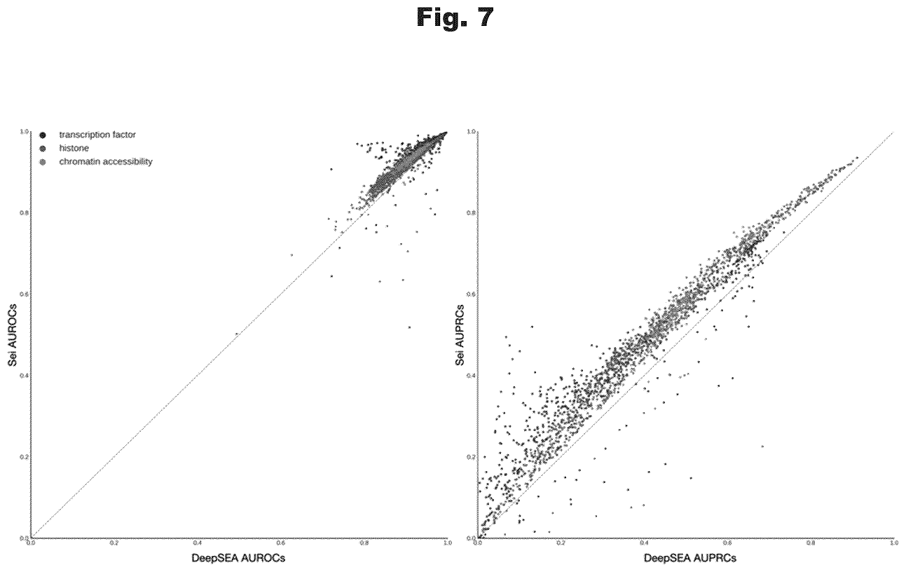

The model achieved an average area under the receiver-operating characteristic (AUROC) of 0.972 and average area under the precision-recall curve (AUPRC) of 0.409 across all 21,907 chromatin profiles (FIG. 5). In addition to accurately predicting individual profiles, the predictions also recapitulated the correlation structure of these profiles, which indicates that the Sei model is able to capture the co-localization patterns of chromatin profiles (FIG. 6). Sei model also improved over the previously published model, DeepSEA “Beluga” (J. Zhou, et al. Nat. Genet. 50, 1171-1179 (2018), the disclosure of which is incorporated herein by reference), on the 2002 chromatin profiles predicted by both models by 19% on average (as measured by AUROC/1-AUROC, FIG. 7).

Therefore, the Sei model is the most comprehensive chromatin-level sequence model to date, and offers an expansive new resource for sequence and variant interpretation.

### Defining Sequence Classes Using a Sequence Model Form Whole Genome Sequences

Next, the Sei model was applied to develop a global, quantitative map from genomic sequences to define distinct classes of regulatory activities (referred to as sequence classes) by leveraging the wide range of transcription factors and histone modifications in the 21,907 Sei chromatin profile predictions. Sequence classes are therefore mapped directly from sequence, and each sequence class represents a distinct program of regulatory activities across all tissues and cell types. Sequence classes allow for the mapping of any sequence to quantitative scores that represent a broad spectrum of regulatory activities.

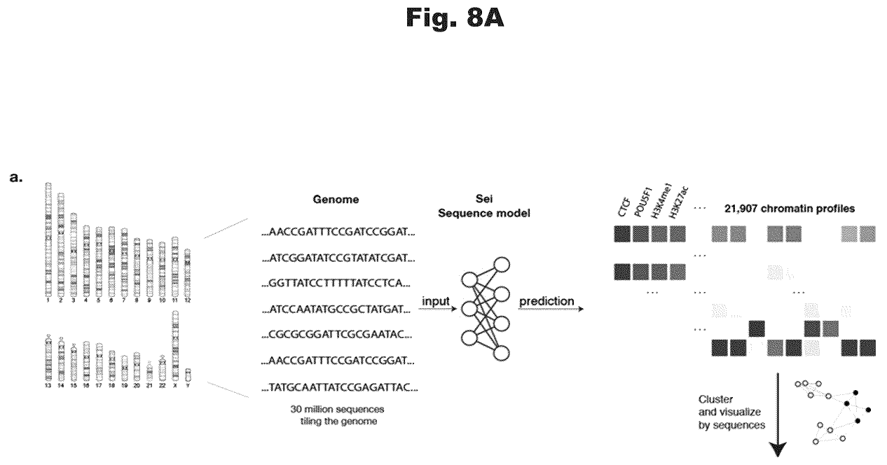

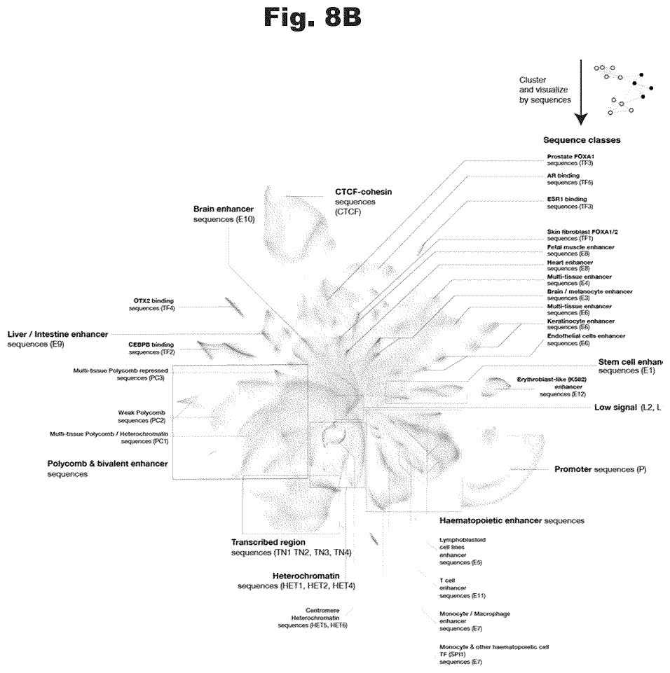

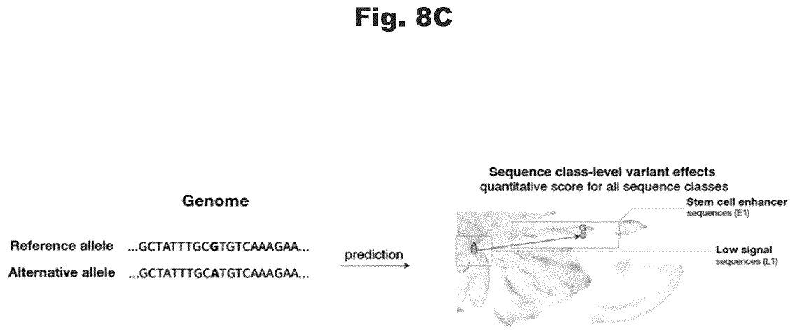

To cover the whole spectrum of sequence activities, sequence classes were identified from Sei predictions for 30 million sequences uniformly tiling the whole genome (4 kb windows with 100 bp step size). The global structure of sequence regulatory signals was visualized as represented by Sei chromatin profile predictions with nonlinear dimensionality reduction techniques (FIGS. 8A-8C; for more on nonlinear dimensionality reduction techniques, see L. McInnes, et al, arXiv:1802.03426 (2018), and P. G. Policar, M. Strazar, and B. Zupan BioRxiv 731877 (2019), the disclosures of which are incorporated herein by reference) and applied Louvain community clustering (V. D. Blondel, et al., J. Stat. Mech. P10008 (2008), the disclosure of which is incorporated herein by reference). to these predictions to categorize the 30 million sequences into 40 sequence classes (FIG. 8A).

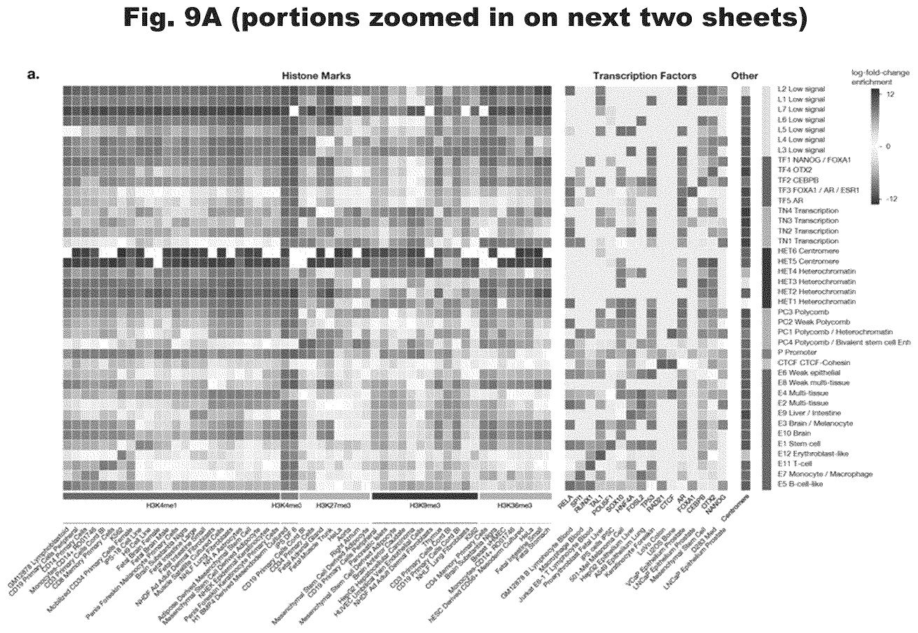

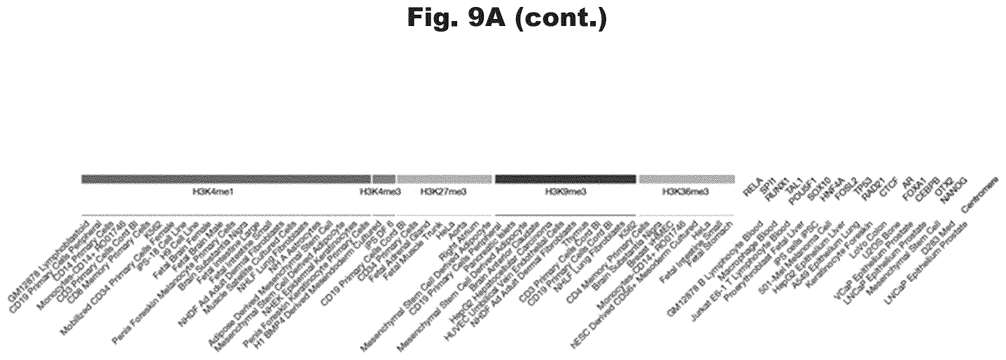

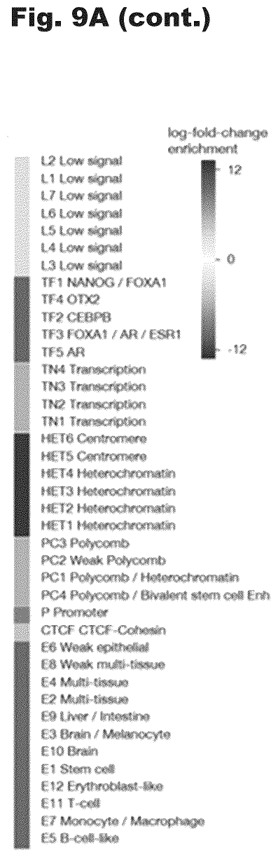

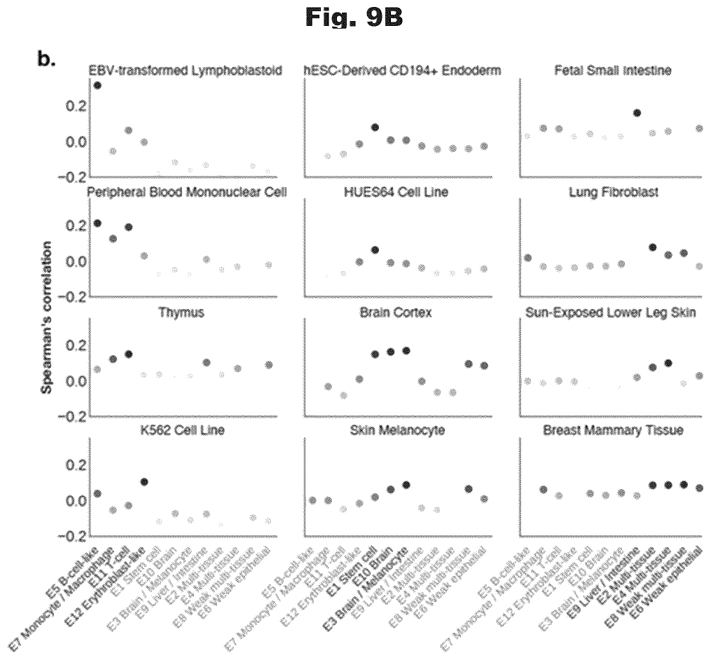

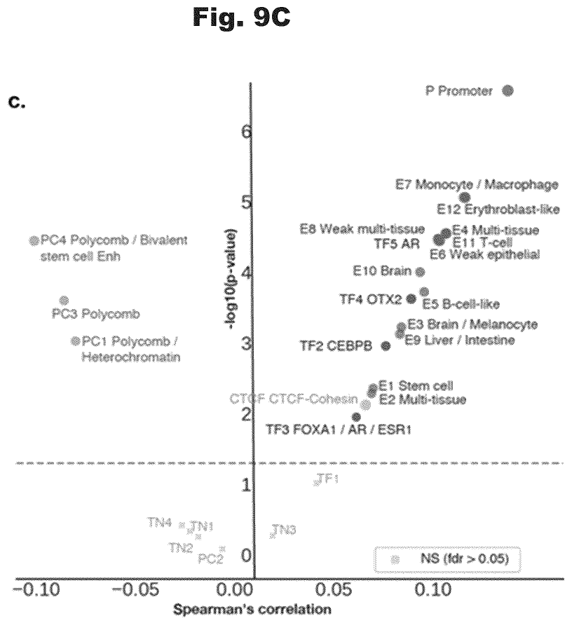

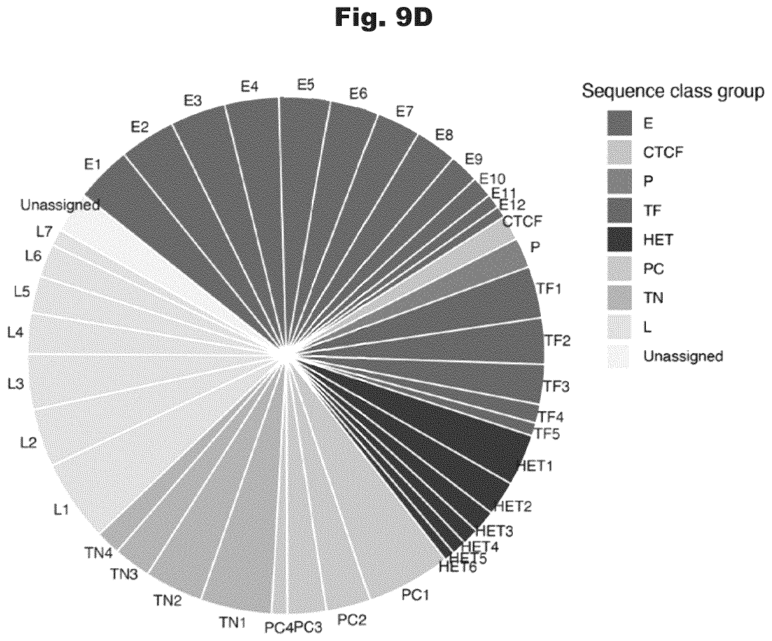

This visualization of human genome sequences demonstrates the global organization of sequence regulatory activities (FIGS. 8B, and 9A-9C). The center of the visualization contains sequences with weak or no regulatory activity based on histone mark and TF enrichment, and sequences with specific regulatory activities radiate outwards, establishing a continuum from no activity to strong specific activity. Different branches of sequences are enriched in distinct chromatin modifications and transcription factors, and sequences with similar regulatory activities are grouped together. For example, tissue-specific enhancer sequences were predominantly grouped by tissue in the visualization (FIG. 8B). In addition, sequences with repressive Polycomb marks were spatially adjacent to H3K9me3-marked heterochromatin sequences (see FIGS. 8A-8C). Notably, promoter-proximal sequences and CTCF-cohesin binding sequences form two well-defined clusters that are separated from other sequences, which may reflect the distinct nature of these activities.

Sequence classes identified from whole genome sequences recapitulate the sequence organization shown in the visualization and provide a basis for summarizing sequence activities globally. To facilitate intuitive interpretation of sequence classes, sequence classes were named based on the enrichment of cis-regulatory profiles (FIG. 9A); specifically, labels were created with a functional group acronym and an index denoting the rank of the sequence class within the group (FIG. 9D) (e.g., E1 encompasses a larger proportion than E2). Because genomic sequences encode their regulatory activity programs across all cell types, sequence classes also show distinct activity patterns across cell types and tissues. We label sequence classes primarily based on their active, cell-type-specific regulatory activities—in particular, promoter and enhancer activities. Therefore, sequence classes that are not labeled as enhancer (‘E’) or promoter (‘P’) generally lack enhancer or promoter activity in any cell type predicted by Sei.

In summary, sequence classes contain 1 ‘P’ promoter class, which is most strongly enriched in the active promoter histone mark H3K4me3 across all cell types (FIG. 9A); 12 ‘E’ enhancer classes, which are strongly enriched in enhancer histone marks, such as H3K4me1 and H3K27ac, and transcription factors relevant to their activities in select cell types (e.g. PU.1/Spi1 in the E7 monocyte/macrophage enhancer class, HNF4-α in E9 liver/intestine, and Sox2/Nanog/Pou5f1 in E1 stem cell), and often display repressive H3K27me3 marks in inactive cell types (FIG. 9A); 1 ‘CTCF’ sequence class, which is strongly enriched in CTCF and cohesin (FIG. 9A); 5 ‘TF’ sequence classes, which are enriched in a few specific transcription factors (e.g. CEBPB sequence class) but have weak or no enhancer mark enrichment; 4 ‘PC’ Polycomb classes, which are enriched in the Polycomb-repressed region mark H3K27me3 and generally not enriched in active promoter or enhancer marks (FIG. 9A); 6 ‘HET’ heterochromatin classes, which are enriched in the heterochromatin mark H3K9me3 (FIG. 9A); 4 ‘TN’ sequence classes, which are enriched in transcription elongation marks H3K36me3 or H3K79me2; and finally, 7 ‘L’ (low signal) sequence classes, which are not strongly enriched in any of the above marks (FIG. 9A). As a whole, the 40 sequence classes cover >97.4% of the genome (FIG. 9D).

Beyond classifying genomic sequences to sequence classes, we define sequence class scores to provide a global and quantitative representation of sequence regulatory activities. This for the first time allows (1) prediction of the regulatory activity for any sequence and (2) quantification of the changes in regulatory activity caused by any sequence variant. Sequence class scores summarize predictions for all 21,907 chromatin profiles based on weights specific to each sequence class, which are computed by projecting Sei predictions onto unit-length vectors that point to the center of each sequence class. Sequences that score highly for a particular sequence class have high predictions for the chromatin profiles associated with that class. Sequence class scores thus allow for the quantification of the regulatory activity of any sequence, and the impact of a variant which can be represented by the difference between the sequence class scores for the reference and alternative alleles.

### Enhancer Sequence Classes Predict Tissue-Specific Gene Expression

The group of sequences that are likely most impactful to tissue-specific gene expression regulation are the enhancer (‘E’) sequence classes. The association of enhancer sequence class scores with tissue-specific gene expression was assessed.

In the visualization of sequence regulatory activities, sequence classes with different cell type and tissue-specific enhancer activities are localized to distinct subregions (FIG. 8B). ‘E’ sequence classes capture both specific and broad enhancer activities. Based on enhancer mark enrichment, E7 is specific for monocyte/macrophage, E11 is specific for T-cell, and E5 is specific for lymphoblastoid/B-cell-like cell lines, E9 is specific for liver and intestine, E1 class is specific for embryonic stem cells & induced pluripotent stem cells, and E10 and E3 are specific for brain (FIGS. 8A-8C and 9A-9C; all enrichments stated are significant with p<2.2e-16, Fisher's exact test, two-sided). In contrast, broad enhancer sequence classes can either encompass enhancer activity in similar cell types across different tissues, such as fibroblast (E2) and epithelial (E6) cell types, or encompass enhancer activity in many different cell types; for example E4 is enriched in fibroblast, muscle, astrocytes, osteoblast, epithelial, and other cell types. Consistent with their predicted enhancer activities, the coverage of ‘E’ sequence class annotations within 10 kb window to transcription start sites (TSS) are correlated with the differential expression patterns of these genes in the corresponding cell types over the tissue-average (FIG. 9B).

Since sequence class scores provide systematic prediction of variant effects on higher-level regulatory functions, one can estimate whether a given variant diminishes, maintains or increases the enhancer activity of a sequence based on the difference between the sequence class scores for the reference and alternative alleles. Evaluated on GTEx eQTL data (The GTEx Consortium, Science 369, 1318-1330 (2020), the disclosure of which is incorporated herein by reference), it was found that variants predicted to increase the ‘E’ sequence class activity were significantly positively correlated with higher gene expression, whereas those predicted to increase ‘PC’ sequence class activity were significantly negatively correlated with gene expression, consistent with the expected repressive role of ‘PC’ sequence class activities (FIG. 9C). Therefore, sequence classes can distinguish effects of variants on gene expression based on their consequences in regulatory activities.

### Regulatory Sequence Classes are Under Evolutionary Constraints

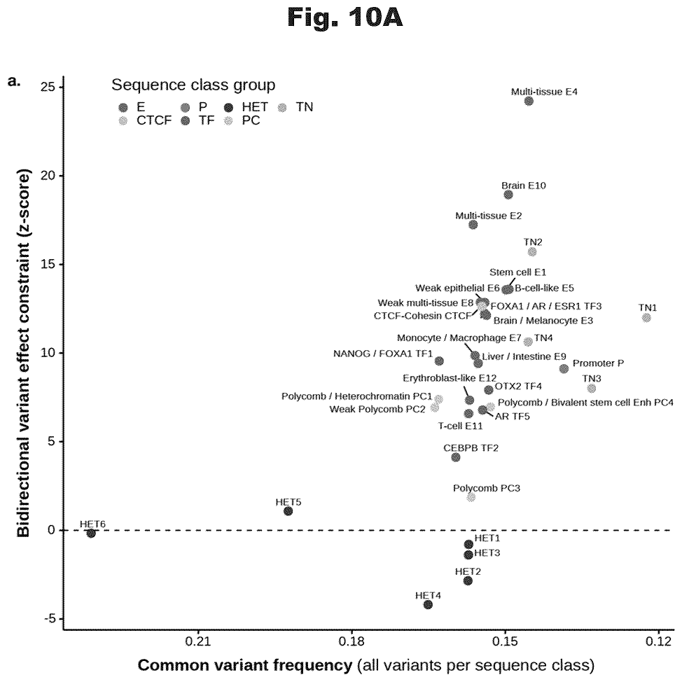

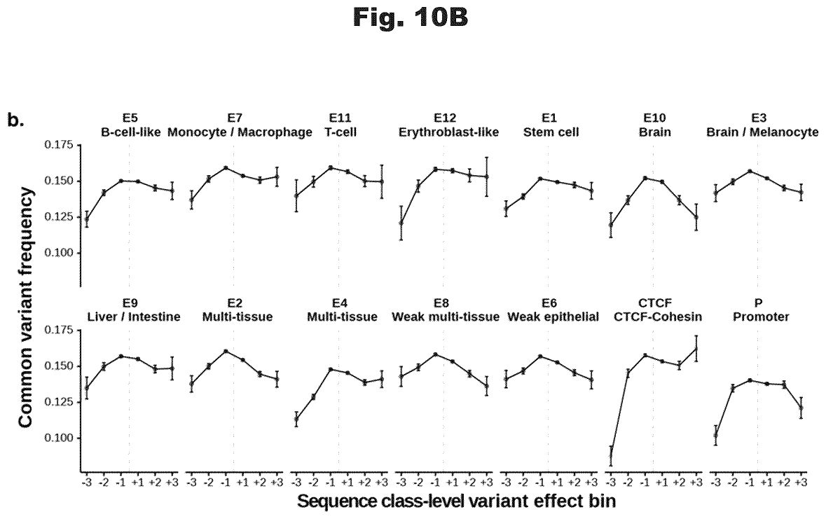

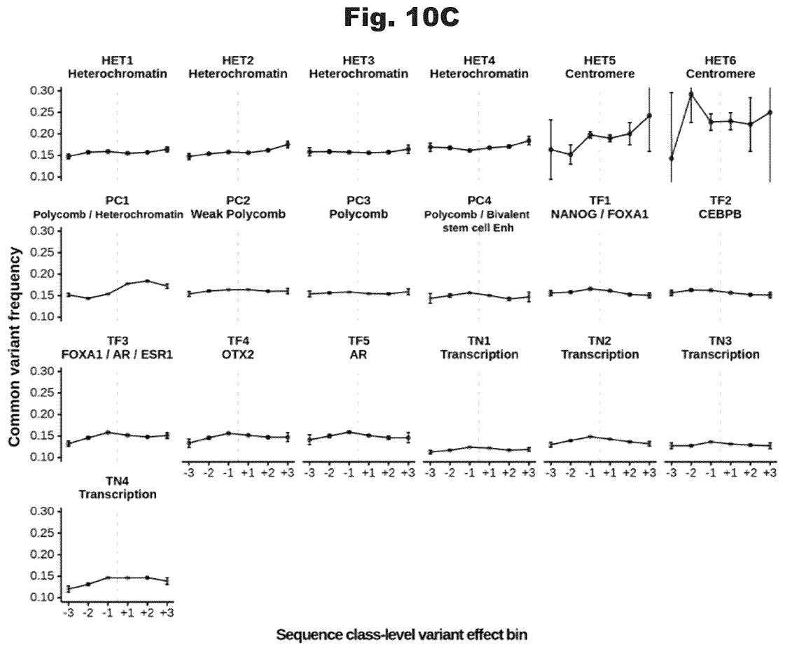

Variants that alter regulatory activities of sequences often disrupt gene regulation and are therefore expected to impact human health and disease. This hypothesis was tested by comparing human population genome variant allele frequencies based on the sequence class in which each variant is located within and the predicted variant effect on that sequence class. Indeed, it was found that variants that localized within E-, P-, and CTCF-sequence classes have lower population allele frequency than variants in other classes (FIG. 10A). More importantly, variants predicted to strongly perturb regulatory sequence classes had significantly lower allele frequencies than variants that weakly perturb these classes (FIG. 10A), consistent with the hypothesis that disruption of regulatory sequence class activities has a major negative impact on fitness, which is referred to herein as a “negative selection signature.

Specifically, strong negative selection signatures was observed for variants assigned to all E, CTCF and P sequence classes (FIGS. 10A and 10B). Multi-tissue enhancer sequence classes E4 and E2 and the brain enhancer E10 show the strongest association of predicted sequence-class-level variant effect and allele frequencies. Notably, for the CTCF sequence class, only negative variant effects—decreasing sequence class activity appears to be under very strong constraints, suggesting that CTCF sites are generally tolerant to positive effect mutations that further increase CTCF binding. This is in contrast to the generally deleterious impact of the increase and the decrease of enhancer and promoter activities. As expected, TN sequence classes, which overlap with protein-coding regions, are among the classes with the lowest allele frequency (FIG. 10C).

In contrast, those assigned to HET, PC, TF, and L sequence classes generally did not show strong negative selection signatures and had higher overall allele frequencies (FIG. 10C). Importantly, this does not suggest that Polycomb or transcription factors are inessential: the HET, PC, TF, and L classes generally do not show strong enhancer or promoter histone mark enrichment in any cell type (with the exception of bivalent marks in stem cells observed in PC4), and thus they are expected to play less major roles in gene expression regulation. However, Polycomb-related regulation is likely critical for ‘E’ and ‘P’ sequence classes, which are often Polycomb-repressed in some cell types but enhancers or promoters in other cell types. Similarly, it is expected that TF binding plays a central role in ‘E’ classes that are highly enriched in relevant TFs (FIG. 9A).

Therefore, sequence classes show distinct evolutionary constraints, and ‘E’ enhancer sequence classes show the strongest bidirectional constraints. This suggests that both increases and decreases of enhancer activity are expected to lead to deleterious effects on fitness, highlighting the importance of precisely controlling gene expression.

### Sequence Classes Elucidate Tissue-Specific Regulatory Architecture of GWAS Traits

The population allele frequency analysis on sequence classes suggests that variants perturbing regulatory sequence class activities are likely involved in human health and disease. Therefore, to explore this hypothesis, GWAS data were used to delineate the genetic contribution of each sequence class to diseases and traits.

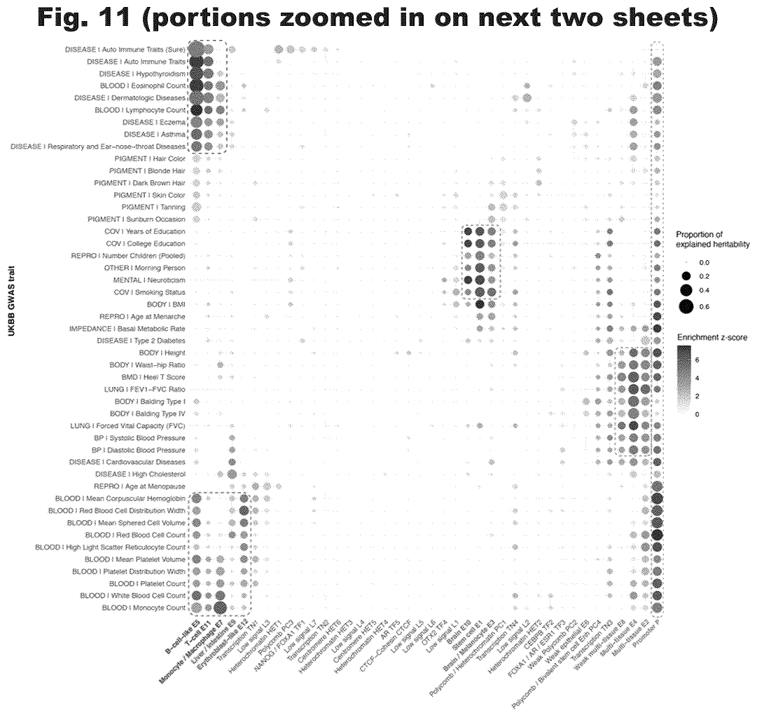

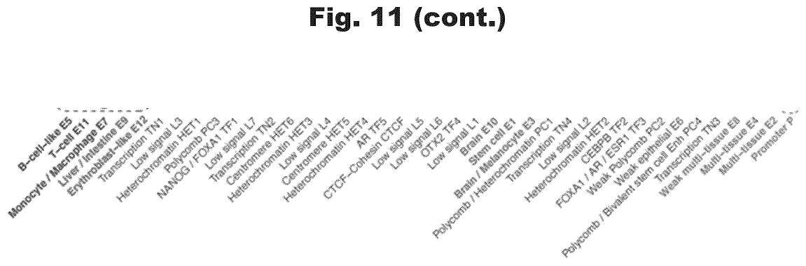

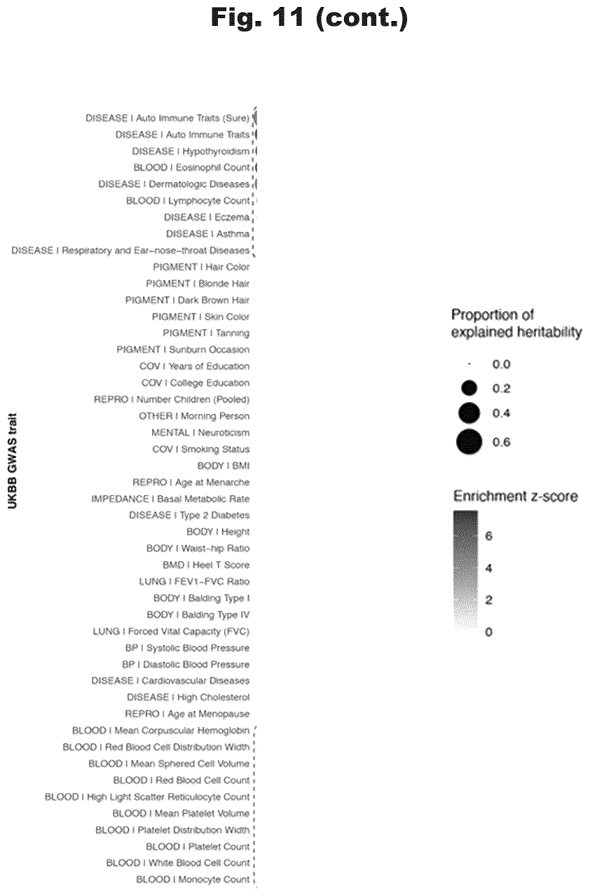

Partitioned heritability from LD score regression (LDSR) has been a powerful tool for understanding the genetic architecture of diseases and traits using GWAS summary statistics (H. K. Finucane, et al. Nat. Genet. 47, 1228-1235 (2015), the disclosure of which is incorporated herein by reference). Previous applications of LDSR use overlapping annotations, and therefore cannot unambiguously partition heritability across annotations. Because sequence classes are both non-overlapping and cover nearly the entire genome, they provide a clear and more easily interpretable picture of the regulatory architecture of diseases and traits. To show this, the proportion of heritability explained by each sequence class was estimated for 47 GWAS traits in UK Biobank (UKBB) (P. R. Loh, et al., Nat. Genet. 50, 906-908 (2018), and C. Sudlow, et al., PLOS Med. 12, e1001779 (2015), the disclosure of which are incorporated herein by reference). Specifically, LDSR was applied using a conservative estimate of the proportion of heritability, subtracting one standard error and lower-bounding by 0. Analysis of UKBB GWAS revealed genetic signatures of sequence-class-specific regulatory functions (FIG. 11).

Remarkably, ‘E’ and ‘P’ sequence classes cover almost all classes that explain a high proportion of heritability for GWAS traits and diseases—the same sequence classes inferred to be under strong evolutionary constraints (FIG. 10A). Three main groups of traits were observed to share similar heritability composition signatures across sequence classes. The first group is blood-related traits, which contains two subgroups of immune-related and non-immune-related traits. The majority of heritability signals in blood-related traits are explained by enhancer classes for the relevant cell type(s), such as monocyte/macrophage enhancer (E7) for Monocyte Count, B-cell-like enhancer (E5) for Auto Immune Traits, and erythroblast-like (red blood cell progenitor) enhancer (E12) for Red Blood Cell Distribution Width, which measures the range of variation in red blood cell volume. Furthermore, autoimmune-related traits are selectively associated with the immune cell type enhancer sequence classes E5 (B-cell like), E11 (T-cell), and E7 (monocyte/macrophage), while erythroblast-like enhancer E12 is specifically linked to red-blood-cell-related traits. Therefore, sequence classes can dissect the cell-type-specific regulatory architecture of traits and diseases with heritability decomposition, even without relying on gene-level information.

The second group of traits with similar sequence-class-level heritability decompositions are cognitive and mental traits (Morning Person, Neuroticism, Smoking Status, Years of Education, College Education), which were mostly explained by brain enhancer (E10 and E3) and stem cell enhancer (E1) sequence classes. The link to E1 is consistent with our observation that E1 was also moderately enriched for active enhancer mark H3K4me1 in brain cell types (FIG. 9A) and is positively correlated with gene expression in brain tissues (FIG. 9B).

The third group of traits is intriguingly diverse, including Balding, Lung Forced Vital Capacity, Waist-hip Ratio, Height, and Heel T-score. The heritability of these traits is mostly explained by multi-tissue enhancer classes (E4, E2, and E8), which show activity in epithelial cells, fibroblast, muscle, and many other cell types. Enhancer activity across multiple tissues in the body may explain the diverse phenotypes that are associated with these traits.

There are traits with unique heritability patterns that were not included in these groups, however, these traits are also linked to highly relevant sequence classes. For example, the High Cholesterol trait was most associated with the liver and intestine enhancer sequence class (E9), which is consistent with the physiology of cholesterol metabolism and known etiology of this condition. E9 was also linked to red-blood-cell-related traits, in line with the role of liver in erythropoiesis.

Finally, the promoter sequence class P uniquely explained a sizable proportion of heritability in nearly all traits, suggesting near-universal involvement of promoter sequence variations in all traits and diseases.

It was next assessed whether the sequence classes could explain GWAS heritability beyond that explained by annotations discovered in prior studies. To this end, LDSR analysis was performed with whole genome annotations of sequence classes conditioned on an up-to-date set of previously identified baseline annotations (v2.2, https://alkesgroup.broadinstitute.org/LDSCORE/). The analysis uncovered 83 significant sequence-class-trait associations with a corrected p-value cutoff of <0.05. 70% of all UKBB GWAS traits and most of the E and P sequence classes (9/13) have at least one significant association after multiple hypothesis testing correction. This finding suggests that sequence classes can identify extensive new regulatory signals that enrich GWAS interpretation.

### Disease Mutations are Predicted to Disrupt the Activities of Sequence Classes

Sequence-class-level effects enable the prediction of specific regulatory mechanisms at the individual, pathogenic mutation level. To showcase the framework's capability to predict the mechanisms of individual mutations, Sei was used to predict the direction and magnitude of sequence-class-level mutation effects for all 853 regulatory disease mutations from the Human Gene Mutation Database (HGMD) (P. D. Stenson, et al., Genome Med. 1, 13 (2009), the disclosure of which is incorporated herein by reference). For systematic classification and quantification of these mutations, each mutation was assigned to an affected sequence class based on its mutation effects (the sequence class with the strongest score change) and the sequence that it alters.

Overall, the average variant effect score of disease mutations is 4.2× larger than the de novo mutations in healthy individuals (0.903 vs 0.217, p<2.2e-16, Wilcoxon rank-sum test two-sided, max absolute effect across sequence classes) and 6.5× larger than the 1000 Genomes common variants with AF>0.01 (0.903 vs 0.139, p<2.2e-16). The mutations with the strongest predicted effects (>1.1, n=138/853) were further analyzed. The predicted effect refers to the variant effect of the assigned sequence class for each mutation. Because sequence-class-level variant effects are directional—that is, predicting whether the alternative allele increases or decreases sequence-class level activity—it was discovered that while the majority (˜80%) of the analyzed pathogenic mutations are predicted to decrease sequence class activity, the remaining 20% of analyzed pathogenic mutations are predicted to increase sequence class activity. Moreover, perturbations to E-, P-, and CTCF-classes make up >99% of the mutations with strong predicted effects on sequence class activity: 44.9% are predicted to affect tissue-specific E sequence classes, 38.4% are predicted to affect the P promoter sequence class, and interestingly, 15.9% are predicted to affect the CTCF-cohesin sequence class.

It was found that almost all mutations with strong predicted effects in cell-type-specific E sequence classes contributed to diseases relevant to that same cell type (FIG. 12)—for most of these mutations, the nearby gene is known to be relevant to the disease but the molecular mechanisms of regulatory disruption are unknown. For example, mutations causing Protein C deficiency and Hemophilia B, two diseases characterized by the deficiency of specific plasma proteins produced in the liver (protein C and coagulation factor IX, respectively), are predicted to decrease E9 liver/intestine sequence class activities. Blood cell-type-specific enhancer sequence classes are disrupted in distinct blood-related diseases and deficiencies relevant to the corresponding cell type: the E12 erythroblast-like enhancer sequence class is disrupted in red-blood-cell-specific diseases such as pyruvate kinase deficiency, erythropoietic porphyria, delta-thalassemia, and beta-thalassemia; the E7 monocyte/macrophage-like sequence class is disrupted in monocyte and macrophage-related chronic granulomatous disease; and the E5 B-cell-like enhancer sequence class is disrupted in X-linked agammaglobulinemia, a functional deficiency of B-cell. For developmental diseases, such as preaxial polydactyly triphalangeal thumb and radial ray deficiency and triphalangeal thumb-polysyndactyly syndrome, the E1 embryonic-stem-cell-specific enhancer sequence class is predicted to be disrupted by mutations in a known distal enhancer of Sonic Hedgehog (SHH) (chr7:156583951 G>A, chr7:156583949 G>C), a gene that plays a crucial role in the positioning and growth of limbs, fingers, and toes during development.

In addition, 38% of the regulatory mutations with strong predicted effects affect the activity of the promoter sequence class P, including a hypercholesterolemia mutation near the LDLR gene (chr19:11200089 C>T), a microcephaly & developmental delay mutation near the PIGY gene (chr4:89444948 C>T), and a retinoblastoma mutation near the RB1 gene (chr13:48877851 G>T). The high proportion of mutations perturbing the P sequence class likely reflects both the critical role of promoters in diseases and the emphasis on promoter-proximal mutations in past studies.

While the mutations discussed thus far are negative effect mutations that decrease sequence class activity, 20% of HGMD pathogenic mutations are predicted to increase sequence class activity. Indeed, these mutations included many known gain-of-function mutations, which validated the predictions. The top positive effect was observed for a mutation (chrX:73072592 G>C) near the XIST gene that skews X-inactivation of the mutant chromosome in females; this mutation was predicted to increase the activity of the CTCF sequence class and has been experimentally validated to increase CTCF binding. Positive effect predictions for ‘E’ and ‘P’ sequence classes were also validated by previously studied mutations: an alpha-thalassemia mutation near the HBM gene (chr16:209709 T>C) known to create a GATA1 binding site and increase intergenic transcription was predicted to increase the activity of erythroblast-specific E12 sequence class, and a TERT gene mutation found in individuals with familial melanoma (chr5:1295161 T>G) was predicted to increase the activity of P. Beyond this, many mutations predicted to have strong positive effects were not previously understood. For example, a mutation near the HBG1 gene (chr11:5271262 A>G) that causes persistence of fetal hemoglobin is also predicted to increase the activity of the erythroblast-specific E12 sequence class. Previously, this mutation was known to create an ATGCAAAT octamer that matches the POU family transcription factor motif, but its functional consequences were unclear.

Notably, even though pathogenic mutations from prior genetics studies are subjected to selection bias, the observation of pathogenic mutations with strong impacts on E-, P-, and CTCF-sequence classes are consistent with the estimation that regulatory sequence classes are under strong evolutionary constraints, and strong disruptions to these classes are likely to cause health consequences. It is also noted that the pathogenic mutations with strong positive effects on the CTCF class do not contradict the population allele frequency analysis which inferred that further increase of activity on the CTCF sequence class is generally tolerated, because all these pathogenic mutations are located within sequences in the other sequence classes, but their sequence class identities are altered to the CTCF class by these mutations. This is in contrast to the allele frequency analysis, which only focuses on variants located in sequences in the CTCF sequence class.

Therefore, sequence-class-level effects both corroborate existing regulatory mechanisms and propose new mechanisms for individual pathogenic mutations. The Sei framework is a valuable tool in accelerating genetic discoveries of disease-causal mutations and their mechanisms in the regulatory genome.

### Analysis of Data Results

A genome-wide sequence-based map of regulatory activities using sequence classes was developed, which is a “vocabulary” of genomic sequences discovered using a data-driven, systematic method. The deep learning-based framework uses a compendium covering 21,907 publicly available cis-regulatory profiles and the whole genome sequence to create a mapping from any sequence to a comprehensive set of sequence classes. This provides a global sequence-based view of sequence regulatory activities and allows for the quantitative prediction of variant effects on sequence class activities. Sequence classes aim to be a concise vocabulary that is interpretable, quantifiable, and easily analyzed both individually and in combination with other sequence classes. It is the first such attempt to our knowledge to systematically map regulatory activities from any sequence.

E- and P-sequence classes are strongly enriched in trait and disease GWAS heritability and under evolutionary constraints. Importantly, sequence classes provide insights into the mechanisms of individual pathogenic mutations by predicting effects on the function of tissue-specific enhancers, promoter activity, and long-range genome interactions (e.g. CTCF-cohesin sequence class). Using sequence-class-level variant effect predictions, many pathogenic mutations were linked to tissue-specific regulatory changes in the relevant tissues. These predictions point to potential mechanisms that can be experimentally tested.

Sequence classes leverage a sequence model trained on most publicly available cis-regulatory profile data; however, there remains substantial space for improvement as more data becomes available. For example, data are lacking for many cell types, developmental stages, transcription factors, and combinations of chromatin targets measured in new cell types or conditions. More data will likely enable the characterization of still more cell-type-specific and developmental stage-specific sequence classes.

This work demonstrates the potential of sequence classes to discover regulatory disruptions in human diseases, through both the aggregation of genome-wide variant association signals and prediction of the impact of individual mutations. Sequence classes and the Sei model are described as an exemplary resource for further understanding the regulatory genetic landscape of human health and diseases. The Sei framework is applicable to any variant, regardless of whether it is common, rare, or never previously observed. Sei is a powerful tool for understanding the mechanistic effects of noncoding mutations in human health.

### Methodology Utilized to Obtain Data Results

### Training Data

21,907 cis-regulatory profiles in peak format were compiled from the processed files in the Cistrome, ENCODE, and Roadmap Epigenomics projects. The Cistrome Project, which systematically processed publicly available cis-regulatory profiles, contributed the majority of the profiles predicted in Sei (19,905). Profiles from Cistrome with less than 1000 peaks were excluded. Genome sequences are from the GRCh38/hg38 human reference genome.

### Deep Learning Sequence Model Training

The Sei model is trained to predict 21,907 transcription factor binding, histone marks, and DNA accessibility from cis-regulatory profile peaks at the center of 4 kb sequences.

The model architecture is composed of three sequential sections: 1) a convolutional network with dual linear and nonlinear paths, 2) residual dilated convolution layers, 3) spatial basis function transformation and output layers. In the convolutional architecture, a new design was introduced, which was composed of both linear and nonlinear convolution blocks. The nonlinear blocks are composed of convolution layers, batch normalization layers and rectified linear activation functions (ReLU), similar to regular convolutional networks. The linear blocks have the same structure as the nonlinear blocks but do not include activation functions to facilitate learning of linear dependencies. Each nonlinear block is stacked on top of a linear block with a residual connection adding the input of the nonlinear block to the output, allowing the computation to go through either the linear or nonlinear path. Dilated convolutional layers with residual connections further expands the receptive fields without reducing spatial resolution. Finally, spatial basis functions are used to reduce dimensionality of the spatial dimension while preserving the capability to discriminate spatial patterns of sequence representations. Specifically, in the Sei model, a B-spline basis matrix (256×16) with 16 degrees-of-freedom across 256 uniformly-spaced spatial bins is generated and multiplied with the convolutional layers output to reduce the 256 spatial dimensions to 16 spline basis function dimensions. After the spline basis function transformation, a fully-connected layer and an output layer are used for integrating information across the whole sequence and generating the final 21,907-dimensional predictions.

Training, validation, and testing datasets are specified by different sets of chromosomes in the hg38 genome (holding out chromosome 8 and 9 for the test set and chromosome 10 for the validation set), and samples drawn uniformly across the hg38 genome for these partitions, excluding regions specified in the ENCODE blacklist. For training, we sampled training sequences and their labels on-the-fly from the training set of chromosomes using Selene (K. Chen, et al. Nat. Methods 16, 315-318 (2019), the disclosure of which is incorporated herein by reference). As a result, almost all training samples are drawn from unique genomic intervals with distinct start and end positions to reduce overfitting during the training process. For each 4 kb region, a 21,907-dimensional binary label vector is created for the 21,907 cis-regulatory profiles based on whether the center basepair overlaps with a peak in each of the profiles. The model is implemented in PyTorch and trained with Selene.

### Model Performance

The AUROC and AUPRC were computed for all cis-regulatory profiles predicted by Sei on the test holdout dataset, excluding profiles that had fewer than 25 positive samples in the test set. Additionally, to assess the correlation structure of the predictions, the rank-transformed pairwise Spearman's rank correlations were compared for the predicted cis-regulatory profiles to the pairwise correlations for the true labels (peak calls provided in Cistrome DB).

The model performance comparison between DeepSEA and Sei is computed on the 2,002 cis-regulatory profiles from Roadmap and ENCODE that both DeepSEA and Sei predict. Because both models have the same chromosomal test holdout (chr8 and chr9), the regions specified in the DeepSEA test holdout set were used to create a common test dataset of sequences and labels on which to evaluate the models.

### Sequence Classes

30 million genomic positions that uniformly tile the genome with 100 bp step size were selected and then Sei computed predictions for 4 kb sequences centered at each position. Sequences overlapping with ENCODE blacklist regions or assembly gaps (“N”s) are removed. To process the 30 million×21,907 predictions matrix, the dimensionality is first reduced with principal component analysis (PCA). The PCA transformations were fitted with incremental PCA using a batch size of 1,000,000 for one pass of the whole dataset, and genomic positions were randomly assigned to batches. The top 180 principal components, scaled to unit variance, were used for constructing a nearest neighbor graph where each node is connected to its k-nearest neighbors by Euclidean distance (k=14). Louvain community clustering with default parameters was applied to the nearest neighbor graph with the python-louvain package, which resulted in 61 clusters. The largest 40 clusters are referred to as “sequence classes” and exclude the remaining (smallest) 21 clusters, which constitute <2.6% of the genome, from the analyses due to their size. This cluster assignment to sequence classes at 100 bp resolution is referred to as sequence class annotations. The genome-wide predictions were visualized by computing UMAP embedding with a subsample of PCA-transformed Sei predictions of 30 million sequences, and the visualization was fine-tuned with OpenTSNE.

### Sequence Class Scores

Each sequence class is represented as a unit vector in the 21,907-dimensional cis-regulatory profile space, in the direction of the average prediction of all sequences assigned to this sequence class among the 30 million. In more formal notation, the vector for sequence class i is

$${v}_{i} = \frac{\overline{{p}_{s ∈ Sequence class i}}}{\overline{{‖ {p}_{s ∈ Sequence class i} ‖}_{2}}} ,$$

where pₛ represents the 21,907-dimensional Sei prediction for sequence s. Each Sei prediction can then be projected onto any sequence class vector to obtain a sequence class-level representation of the prediction, which we call sequence class score or score_{s,i}=pₛ·vᵢ^{T}. In addition, predicted sequence-class-level variant effects are represented by the difference between the sequence class scores of the sequences carrying the reference allele and the alternative allele, or score_{v,i}=score_{alt,i}−score_{ref,i}. To better represent predicted variant effects on histone marks, it is helpful to normalize for the nucleosome occupancy (e.g. loss-of-function mutation near TSS can decrease H3K4me3 modification level while increasing nucleosome occupancy, resulting in an overall increase in observed H3K4me3 quantity). Therefore, for variant effect computation, the sum of all histone profile predictions was used as an approximation to nucleosome occupancy and adjust all histone mark predictions to remove the impact of nucleosome occupancy change (non-histone mark predictions are unchanged):

$${p}^{hm *} = {p}_{ref}^{hm} \frac{{\sum }_{k} {p}_{ref}^{{hm}^{k}} + {\sum }_{k} {p}_{alt}^{{hm}^{k}}}{{\sum }_{k} {p}_{ref}^{{hm}^{k}}} ; {p}^{hm *} = {p}_{alt}^{hm} \frac{{\sum }_{k} {p}_{ref}^{{hm}^{k}} + {\sum }_{k} {p}_{alt}^{{hm}^{k}}}{{\sum }_{k} {p}_{alt}^{{hm}^{k}}}$$

where

$${\sum }_{k} {p}_{ref}^{{hm}^{k}}$$

represents the sum over all histone mark predictions (among 21907-dimensions of a prediction) for the reference allele. Low Signal sequence classes were generally excluded in sequence-class-level variant effect analyses because they lack an intuitive biological interpretation.

### Sequence Class Enrichment of Histone Mark Annotations, Transcription Factors, and Repeats

The log fold change enrichment of various chromatin profiles was computed and genome annotations for each sequence class based on sequence class annotations (described above, see ‘Sequence classes’). Log fold change enrichment is computed by taking the log ratio of the proportion of a sequence class intersecting with the annotation versus the background proportion of the annotation, where all regions assigned to any sequence class were considered. Enrichment was computed for all 21,907 profiles predicted by Sei, filtered the chromatin profiles for each sequence class to only those having Benjamini-Hochberg corrected p-values (Fisher's exact test, two-sided) below 2.2e-16, and selected the top 25 profiles based on log fold change enrichment. Cistrome Project profile enrichment is computed over 2 million random genomic positions.

The annotation of centromere repeats is obtained from the UCSC RepeatMasker track, and annotations of histone marks over multiple cell types are obtained from the Roadmap Epigenomics project—enrichments for both of these sets of annotations are computed over the entire genome.

Enhancer Sequence Class Correlations with Cell-Type-Specific Gene Expression

Tissue expression profiles are from GTEx, Roadmap Epigenomics, and ENCODE and transformed to log-scale RPKM (reads per kilobase per million reads mapped) scores and normalized by tissue-average. Specifically, a pseudocount was added before log transformation (0.0001 for GTEx tissues which are averaged across individuals and 0.01 for Roadmap and ENCODE tissues). After log transformation, the average scores across tissues were subtracted for each gene; as a result, the processed scores represent log fold change relative to tissue-average.

Gene-wide expression prediction is evaluated on sequence class annotations (from Louvain community clustering) for positions within +/−10 kb of the TSSs for these genes. For each enhancer sequence class and tissue, the Spearman correlation was computed between the sequence class annotation coverage and gene expression.

Correlation Between Regulatory Sequence Class Variant Effects and Directional eQTL Variant Effect Sizes

The eQTLs within +/−5 kb of gene TSSs were collected from GTEx v*, combined across all GTEx tissues, and computed the Spearman correlation between the top 15 k variant effect predictions for each sequence class and the eQTL variant effect sizes (averaged across multiple tissues if the variant is an eQTL in multiple tissues). The p-values are derived from the Spearman's rank correlation test (two-sided) and BH correction is applied. Low Signal and Heterochromatin sequence classes are excluded from this analysis due to lack of interpretation for their variant effect scores in this context.

### Evolutionary Constraints on Variant Effects

Sequence-class-level variant effects were computed for all 1000 Genomes project phase 3 variants (1000 Genomes Project Consortium, et al. Nature 491 56-65 (2012), the disclosure of which is incorporated herein by reference). Variants are assigned to sequence classes based on the 100 bp resolution genome-wide assignment derived from Louvain community clustering as described above. For each sequence class we divide variants into 6 bins based on their effects in the same sequence class as illustrated in FIG. 3, and summarize common variant (AF>0.01) frequencies in each bin by mean and standard error of the mean. Statistical significance of allele frequency dependency on sequence-class-level variant effects was also estimated. For each sequence class, logistic regression was applied separately for positive effect and negative effect variants, to predict common variants (AF>0.01) from the absolute value of sequence-class-level variant effect score, and obtained the significance z-score of the regression coefficient of variant effect. The bidirectional evolutionary constraint z-score is defined as the negative value of the combined z-scores from positive and negative effect variants with Stouffer's method.

### Partitioning GWAS Heritability by Sequence Classes

UKBB GWAS summary statistics were obtained from P. R. Loh, et al., cited supra. To study the association of sequence class genome annotation and sequence class variant effects and trait heritability, partitioned heritability LD score regression (LDSR) was performed as described in H. K. Finucane, et al., cited supra. To partition the heritability as sums of heritability explained by each sequence class, LDSR was run with only sequence class annotations and a baseline all-ones annotation. The estimated proportion of h² explained by each sequence class and its standard error was obtained with LDSR. As the estimated proportions can have high variance or even be negative, a conservative estimator of the estimated proportion of h² subtracted by 1 standard error was used with a lower-bound of 0. To assess the contribution of sequence classes to explaining additional heritability when conditioned on known baseline annotations, LDSR was also ran with baseline annotations (v2.2, https://alkesgroup.broadinstitute.org/LDSCORE/). The p-values are derived from the coefficient z-score, and BH correction is applied.

### Sequence Class-Level Variant Effect Analysis of Noncoding Pathogenic Mutations

All mutations assigned “DM” and “regulatory” annotation in the Human Gene Mutation Database (HGMD) database (2019.1 release) were analyzed. RMRP gene mutations are excluded because they are likely pathogenic due to impacting RNA function instead of regulatory perturbations, despite being annotated to the regulatory category in HGMD. For every mutation, the sequence class scores were predicted for both the reference and the alternative allele and computed the sequence-class-level variant effect as the predicted scores for the alternative allele subtracting the scores for the reference allele. To provide an overview of sequence-class level effects of human noncoding pathogenic mutations, mutations are first assigned to sequence classes based on the sequence class annotations of the mutation position. For mutations with a strong effect in a different sequence class than the originally assigned sequence class (absolute value higher than the original sequence class by >1 absolute difference and >2.5 fold relative difference), the mutation was reassigned to the sequence class with the strongest effects.

### Cancer Diagnostics

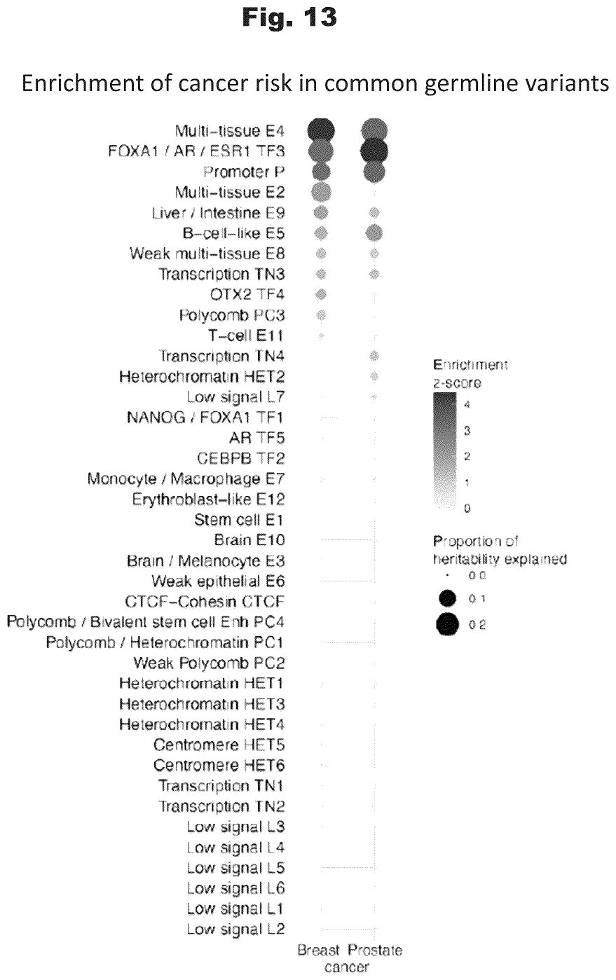

As proof of concept that Sei can impart clinical information on risk of inherited cancers, standard linkage disequilibrium score regression (LDSR) was used to partition heritability of risk, while controlling for LD structure, using Sei impacts of germline noncoding regulatory variants (˜8 million variants). The analysis focused on breast and prostate cancer GWAS because both have large sample sizes and therefore, sufficient power for analysis. When partitioning heritability using Sei sequence classes as annotations for each SNP, significant enrichment of cancer risk in common germline variants was observed for regulatory sequence classes (e.g. promoter, enhancer, specific transcription factor binding), identifying not only that these classes of mutations confer significant risk, but also delineating mechanisms conferring risk (FIG. 13).

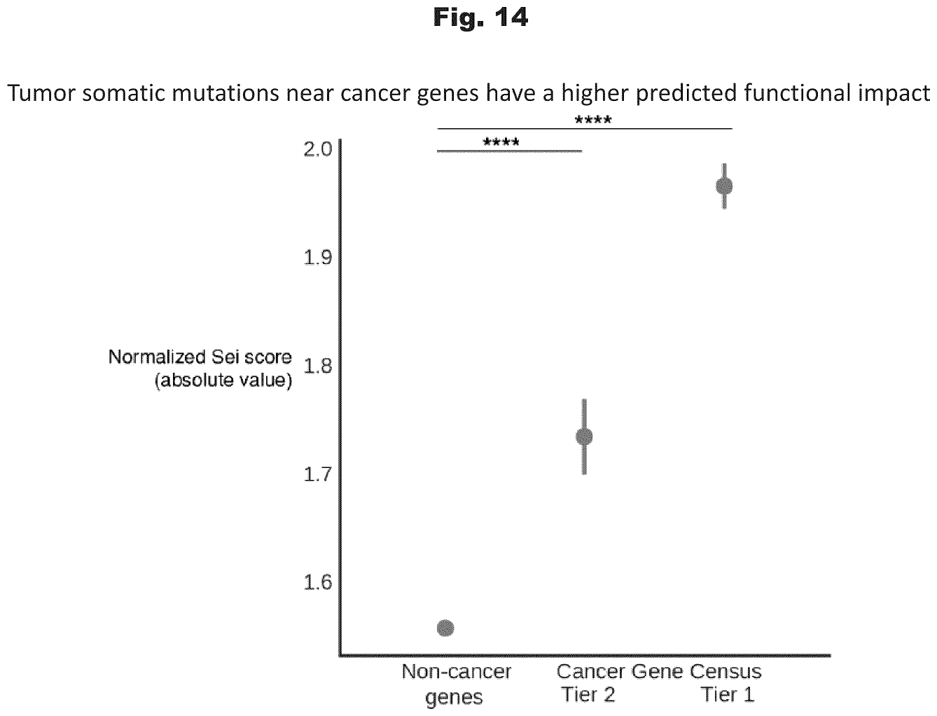

Sei scores were predicted for tumor somatic mutations (approximately 13 million variants) across all cancers in International Cancer Genome Consortium (ICGC) within +/−100 kb of gene transcription start sites (TSS). When comparing the mean and 95% confidence intervals of Sei scores for somatic mutations near non-cancer genes to those near known cancer genes (from COSMIC Cancer Gene Census (CGC)), it was found that higher impact tumor somatic mutations affect cancer genes (FIG. 14). These findings establish that screening regulatory impacts of mutations near cancer genes can find mutations with strong impacts and this can inform clinical decisions, akin to how exon mutation profiles inform patient management. For example, tumors with TERT exon (and promoter) mutations are specifically considered for management of melanoma. The promoter of TERT is recurrently mutated and correlated with gene expression impacts and cancer outcomes. Sei predictions not only identify these mutations as high impact but pinpoint additional mutations in CGC promoters with high impact. This reduces the scale of the problem of studying specific mutations for consideration in clinical management. Sei enables this analysis for highly detrimental rare mutations where statistical approaches fail.

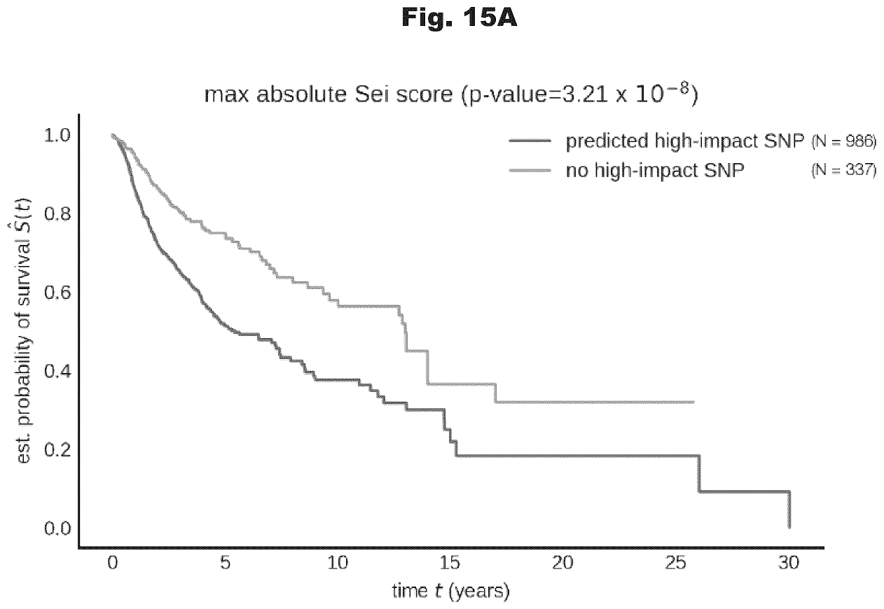

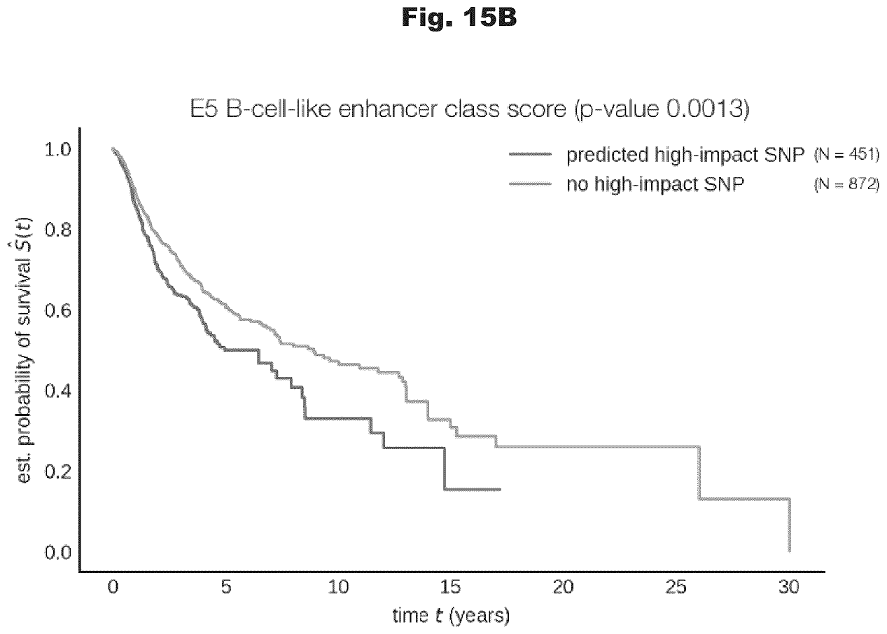

High-impact noncoding tumor somatic variants are predictive of survival. Looking at Sei scores for ICGC pan-cancer somatic mutations within 100 kb of Cancer Gene Census gene TSSes, patients with at least 1 tumor mutation with any Sei sequence class score above 1.1 (threshold determined by Sei predictions for HGMD pathogenic variants, N=986/1323) had poorer survival outcomes than patients without a high impact Sei score (FIG. 15A). Performing more specific analysis in the E5 B-cell like enhancer class score also reveals that patients with high impact scores n this sequence class also have poorer survival outcomes (FIG. 15B). This analysis can aid patient stratification for prioritizing treatments or research directions and further informs these choices by providing possible mechanistic underpinnings of dysregulation that may be exploited for patient benefit.

1.-43. (canceled)

44. A method to assess a regulatory effect of one or more unannotated variants of a biological sample derived from an individual, the method comprising:
obtaining genetic sequence data of an individual;
identifying one or more variants within the genetic sequence data; and
predicting, using a computational framework, a regulatory effect of the one or more variants on transcription of one or more genes, wherein the predicted regulatory effect of the one or more variants on one or more genes informs a diagnosis of the individual.

45. The method of claim 44, wherein the prediction of the regulatory of the one or more variants on transcription of one or more genes comprises:
obtaining a set of one or more classes of genetic sequences, wherein each class of genetic sequences comprises a plurality of genetic sequences each having a computed class sequence score;
obtaining a first genetic sequence;
obtaining a second genetic sequence comprising the one or more genetic variants as compared to the first genetic sequence, wherein the second genetic sequence is derived from the genetic sequence data of the individual, and wherein the first genetic sequence and the second genetic sequence have the same sequence flanking the positions of the one or more variants;
yielding a sequence class score for the first genetic sequence and a sequence class score for the second genetic sequence, wherein each sequence class score is computed via a computational framework that predicts cis-regulatory effects of each genetic sequence, then dimensionally reduces and clusters the predicted cis-regulatory effects into a class of the one or more classes of genetic sequences, and then computes a sequence class score based on the dimensionality reduction and clustering; and
comparing the sequence class score for the second genetic sequence comprising one or more genetic variants with a sequence class score for the first genetic sequence.

46. The method of claim 45, wherein the first genetic sequence is a partial human reference genome selected from: GRCh38/hg38, GRCh37/hg19, NCBI36/hg18, NCBI35/hg17, NCBI34/hg16, or a combination thereof.

47. The method of claim 45, wherein the first genetic sequence is a sequence derived from a relative of the individual.

48. The method of claim 45, wherein the first genetic sequence is a sequence compiled from a plurality of individuals.

49. The method of claim 44, wherein the one or more variants comprise one of: a de novo variant, a novel variant, or an unannotated variant.

50. The method of claim 44, where in the diagnosis of the individual is for a complex disorder.

51. The method of claim 50, wherein the complex disorder is one of: autism spectrum disorder, Alzheimer disease, arthritis, asthma, bipolar disorder, cancer, cleft lip and/or palate, coronary artery disease, Crohn's disease, dementia, depression, diabetes (type II), heart disease, heart failure, high cholesterol, hypertension, hypothyroidism, irritable bowel syndrome, obesity, osteoporosis, Parkinson disease, rhinitis (allergic and nonallergic), psoriasis, multiple sclerosis, schizophrenia, sleep apnea, spina bifida, or stroke.

52. The method of claim 50 further comprising:
administering to the individual a treatment for the complex disorder based on the diagnosis.

53.-68. (canceled)

69. A method for determining an individual's heritability risk for a complex disorder, the method comprising:
obtain genetic sequence data of an individual;
identify one or more inherited variants within the genetic sequence data;
predicting, using a computational framework, a magnitude of impact of regulatory effect of the one or more variants; and
combining a heritability risk calculator that uses clinical information with the predicted magnitude of impact of regulatory effect of the one or more variants to yield a heritability risk of a complex disorder.

70. The method of claim 69, wherein the prediction of the magnitude of impact of the regulatory effect of the one or more variants comprises:
obtaining a set of one or more classes of genetic sequences, wherein each class of genetic sequences comprises a plurality of genetic sequences each having a computed class sequence score;
obtaining a first genetic sequence;
obtaining a second genetic sequence comprising the one or more genetic variants as compared to the first genetic sequence, wherein the second genetic sequence is derived from the genetic sequence data of the individual, and wherein the first genetic sequence and the second genetic sequence have the same sequence flanking the positions of the one or more variants;
yielding a sequence class score for the first genetic sequence and a sequence class score for the second genetic sequence, wherein each sequence class score is computed via a computational framework that predicts cis-regulatory effects of each genetic sequence, then dimensionally reduces and clusters the predicted cis-regulatory effects into a class of the one or more classes of genetic sequences, and then computes a sequence class score based on the dimensionality reduction and clustering; and
comparing the sequence class score for the second genetic sequence comprising one or more genetic variants with a sequence class score for the first genetic sequence, wherein the magnitude of impact is determined by the comparison of the class scores.

71. The method of claim 70, wherein the first genetic sequence is a partial human reference genome selected from: GRCh38/hg38, GRCh37/hg19, NCBI36/hg18, NCBI35/hg17, NCBI34/hg16, or a combination thereof.

72. The method of claim 70, wherein the first genetic sequence is a sequence derived from a relative of the individual.

73. The method of claim 70, wherein the first genetic sequence is a sequence compiled from a plurality of individuals.

74. The method of claim 69, wherein the one or more variants comprise one of: a de novo variant, a novel variant, or an unannotated variant.

75. The method of claim 69 further comprising:
identifying transcriptional regulatory regions that contribute to the heritability risk for the complex disorder; and
administering to the individual a treatment regimen for the complex that target the transcriptional regulatory regions that contribute to the heritability risk.

76. The method of claim 69, wherein the heritability risk calculator is a linkage disequilibrium score regression.

77. The method of claim 69, wherein the complex disorder is one of: autism spectrum disorder, Alzheimer's disease, arthritis, asthma, bipolar disorder, cancer, cleft lip and/or palate, coronary artery disease, Crohn's disease, dementia, depression, diabetes (type II), heart disease, heart failure, high cholesterol, hypertension, hypothyroidism, irritable bowel syndrome, obesity, osteoporosis, Parkinson disease, rhinitis (allergic and nonallergic), psoriasis, multiple sclerosis, schizophrenia, sleep apnea, spina bifida, or stroke.
